(19) United States
(12) Patent Application Publication
(10) Pub. No.: US 2026/0026912 A1
(43) Pub. Date: Jan. 29, 2026

(54) METHODS, SYSTEMS, AND COMPUTER READABLE MEDIA FOR EXTENDED REALITY-BASED SYSTEMS FOR SURGICAL NAVIGATION

(71) Applicant: The Trustees of the University of Pennsylvania, Philadelphia, PA (US)

(72) Inventors: Stephen H. Lane, Princeton, NJ (US); Thomas P. Schaer, Landenberg, PA (US); Samir Mehta, Haddonfield, NJ (US); Lennart Meincke, Philadelphia, PA (US); Shubham Sharma, Santa Clara, CA (US)

(21) Appl. No.: 19/281,129

(22) Filed: Jul. 25, 2025

Related U.S. Application Data
(60) Provisional application No. 63/675,649, filed on Jul. 25, 2024.

(51) Int. Cl. A61B 90/00; A61B 17/17; A61B 34/10; G06T 7/73; G06T 19/00

(52) U.S. Cl. CPC A61B 90/37; A61B 17/1703; A61B 17/1717; A61B 34/10; G06T 7/73; G06T 19/006; A61B 2034/102; A61B 2034/105; A61B 2034/107; A61B 2090/365; A61B 2090/376; A61B 2560/0238; A61B 2562/0219; G06T 2207/10121; G06T 2207/30052

(57) ABSTRACT

A surgical navigation system includes an extended reality head-mountable display (HMD). The surgical navigation system further includes a computer system comprising at least one processor, wherein the computer system is configured for performing operations. The operations include receiving camera images while a surgeon is wearing the HMD and performing a surgical procedure on a subject. The operations further include identifying at least one image feature depicted in the images. The operations further include tracking a location of at least one surgical object using the image feature. The operations further include sending one or more visual indicators to the HMD to display, to the surgeon, a target location for the surgical object for the surgical procedure. The target location includes a location external to the subject for the surgeon to use the surgical object to perform the surgical procedure on the subject.

### PRIORITY CLAIM

This application claims the priority benefit of U.S. Provisional Patent Application Ser. No. 63/675,649, filed Jul. 25, 2024, the disclosure of which is incorporated by reference herein in its entirety.

### TECHNICAL FIELD

The subject matter described herein relates generally to methods, systems, and computer readable media for extended reality-based systems for surgical navigation.

### BACKGROUND

Minimally invasive surgical procedures continue to be popular due to their relative simplicity, lower cost, and reduced patient recovery times. Because such procedures generally restrict the surgeon's ability to see the operative area, surgeons generally rely on computer systems, such as computer assisted surgical navigation (CASN) systems to assist in the surgical operations.

Extended reality technologies have opened new possibilities for improving the efficacy of surgical procedures through generation and display of real-time graphics (based on patient-specific medical data) that help guide and assist the surgeon during a procedure. This is especially relevant for many orthopedic surgical procedures and applications. For example, such a system can be used to provide a surgeon with “X-ray vision” by displaying 3D graphical models and other imagery using smart AR glasses (such as the Microsoft HoloLens) illustrating internal surgical anatomy normally hidden to the surgeon. This could also include highlighting pertinent anatomical landmarks and implant placement locations, including safe corridors for drill and screw trajectories associated with the surgical procedure being performed.

### SUMMARY

The subject matter described herein relates to extended reality surgical navigation systems configured for providing real-time intraoperative guidance when placing locking screws into the distal end of intramedullary (IM) nails without the need for concurrent fluoroscopic guidance or for accurate screw placement during minimally invasive plate osteosynthesis. The extended reality approach taken is compatible with existing implant systems, making the systems agnostic to use with commercially available intramedullary nailing solutions, thereby enhancing its utility and commercial versatility. The systems can be used for other types of surgical procedures, e.g., other orthopedic procedures that involve surgical implantation of two or more objects or devices that are joined together or oriented with one another, such as spine, pelvis, recon, and the like.

A surgical navigation system includes an extended reality head-mountable display (HMD), including, but not limited to, a head mountable display with a visor or glasses form factor. The surgical navigation system further includes a computer system comprising at least one processor, wherein the computer system is configured for performing operations. The operations include receiving camera images while a surgeon is wearing the HMD and performing a surgical procedure on a subject. The operations further include identifying at least one image feature depicted in the images using traditional computer vision or machine learning processing techniques. The operations further include tracking a location (i.e., a position and/or orientation) of at least one surgical object using the image feature. The operations further include sending one or more visual indicators to the HMD to display, to the surgeon, a target location for the surgical object for the surgical procedure. The target location includes a location external to the subject for the surgeon to use the surgical object to perform a surgical procedure the subject.

The subject matter described herein may be implemented in hardware, software, firmware, or any combination thereof. As such, the terms “function” or “node” as used herein refer to hardware, which may also include software and/or firmware components, for implementing the feature(s) being described. In some exemplary implementations, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter described herein will now be explained with reference to the accompanying drawings, of which:

FIG. 1 is a block diagram of an example extended reality surgical environment that includes an extended reality head mounted display (HMD) and a computer system configured for providing surgical guidance to a surgeon performing a surgical procedure on a subject;

FIG. 2 is a flow diagram of an example method for extended reality-based systems for surgical navigation;

FIG. 3 is a perspective view of a tibial nail suitable for use with the extended-reality-based surgical navigation system described herein;

FIG. 4 is a perspective view of an insertion handle of a tibial nail insertion system;

FIG. 5 is a perspective view of a drill guide of a tibial nail insertion system;

FIG. 6 illustrates perspective views of an aiming arm and insertion handle of a tibial nail insertion system;

FIG. 7 illustrates an example of adapting smart insertion handle and drill guide to existing hardware components for minimally invasive plate osteosynthesis/less invasive stabilization system (MIPO/LISS);

FIG. 8 is a block diagram illustrating exemplary components of an extended reality surgical navigation system;

FIGS. 9A and 9B respectively illustrate a head-mountable display suitable for use with the extended reality surgical navigation system described herein and a user wearing the head-mountable display;

FIG. 10A shows the display of a mobile device with an annotation (an arrow in this case) added by the mobile device user instructing the extended reality headset wearer to move the drill guide in the direction of the arrow;

FIG. 10B illustrates the view shown on the display of the extended reality headset corresponding to the view shown on the mobile device in FIG. 10A;

FIG. 10C illustrates the display on the mobile device after the extended reality headset wearer has moved the drill guide to the target location;

FIG. 10D illustrates the view shown on the display of the extended reality headset corresponding to the view shown on the mobile device in FIG. 10C;

FIG. 11 is a perspective view of a smart insertion handle assembly;

FIGS. 12A-12C are perspective views of a smart drill guide assembly;

FIG. 13 illustrates exemplary components of a drill guide;

FIG. 14 is a perspective view of a smart drill guide assembly with an additional sensor for determining drilling depth;

FIG. 15 is a perspective view of a smart calibration pin suitable for calibrating the surgical navigation system;

FIG. 16 illustrates an example of a smart screw inserted into an intramedullary nail;

FIG. 17A illustrates an example of a user interface that may be displayed by a desktop application of an extended reality surgical navigation system;

FIG. 17B illustrates exemplary components of the desktop application;

FIG. 17C is a block diagram illustrating exemplary components of scenario manager of the desktop application;

FIG. 18 is a block diagram illustrating exemplary components of a server application of an extended reality surgical navigation system;

FIG. 19 is a block diagram illustrating exemplary components of HMD/mobile device client application of an extended reality surgical navigation system;

FIG. 20 is a flow chart illustrating an exemplary process for an IM nail surgical procedure supported by the extended reality surgical navigation system described herein;

FIG. 21A illustrates an intraoperative fluoroscopic display taken by surgeon in operating room showing image target impervious to X-rays, nail hole locations and change in previously calibrated screw hole location due to nail deflection;

FIG. 21B illustrates a computer screen shot of intraoperative procedure showing X-ray hole verification step to address possible nail deformation;

FIG. 22 illustrates the position of the distal screw hole axis;

FIG. 23 illustrates the position of the screw hole and the drill guide;

FIG. 24 illustrates the moving of the drill guide towards the displayed virtual drill axis;

FIGS. 25 and 26 illustrate positioning the end of the drill axis in the position of the virtual drill axis;

FIG. 27 illustrates orienting the drill guide with the virtual drill axis;

FIG. 28 illustrates the user drilling a hole when the drill guide is aligned with the virtual drill axis;

FIG. 29 illustrates coordinate systems and translations of the coordinate systems used by the surgical navigation system described herein;

FIG. 30 is a schematic diagram of an LPMS sensor and a translation of the coordinate system used by the LPMS sensor;

FIG. 31 is an image of a drill guide illustrating distances used to calculate drill guide position and orientation;

FIG. 32 illustrates images of a test of the surgical navigation system described herein; and

FIG. 33 includes fluoroscopic control images from the test illustrated in FIG. 32.

### DETAILED DESCRIPTION

### Overview

The subject matter described herein includes extended reality-based systems, methods, and devices which generate pre-operative plans along with real-time surgical navigation, direction and feedback that are customized for a particular patient. One such system includes a surgical planning application and an extended reality computer-assisted surgery system. The system can include a client-server desktop application with a graphical user interface, an extended reality head mounted display (HMD) in the form of a visor or glasses worn by the surgeon, and spatially tracked instruments and surgical devices, and the system assists the surgeon while they perform a surgical procedure by providing visual and auditory cues, as well as 3D graphical annotations and other feedback to enable the surgeon to successfully execute a surgical procedure.

The term “extended reality” is used herein to refer to any appropriate technology that overlays virtual elements or digital content onto the real world, enhancing a user's perception and interaction with their environment. It encompasses various forms of mixed reality (MR) systems that blend virtual and real-world elements. The term “extended reality” is intended to include augmented reality and other forms of mixed reality. Extended reality enables users to experience a merged reality where virtual objects and information coexist with the physical surroundings. Mixed reality encompasses a spectrum of experiences ranging from the purely physical (the real world) to the purely virtual (computer-generated environments). MR can be used to create a seamless blend of the real and virtual worlds. In mixed reality, virtual objects are not only overlaid onto the real world but also interact and respond to the physical environment in real time. This interaction enables users to perceive and manipulate virtual objects as if they were part of the real world.

In one example, the desktop application includes operational modes supporting preoperative surgical planning, real-time surgical visualization, including livestreaming and monitoring, and surgical navigation control. The planning mode features of the desktop application enables surgical staff to create a customized surgical plan based on relevant pre-operative patient image data scans obtained from sources, such as computed tomography (CT), ultrasound (US), nuclear magnetic resonance (NMR), etc. as well as select and calibrate surgical implant devices appropriate for the patient. The surgical visualization and monitoring mode allows multiple staff, trainees, and other participants in the operating room or elsewhere (remote) to view and interact with the extended-reality-enhanced surgical procedure from different points of view, perform intra-operative calibration, as well as to provide direction and assistance to the surgeon.

The desktop application also supports data and video recording and logging enabling the procedure to be replayed and/or viewed at a later point in time for training and education purposes. The surgical navigation control mode allows a user of the desktop application to sequence and control the display of surgical guidance graphics and other information appearing in the extended reality head-mounted display worn by the surgeon. The initial surgical application of the system is tibial intramedullary nail (IM) insertion and interlocking. Enhancements and improvements of the system over some conventional systems include: 1) use of self-referencing systems and sensors such as head-mounted computer vision systems and wireless inertial measurement unit (IMU) orientation sensors (i.e., no need to set up external optical camera or electromagnetic sensor systems in the operating room), use of computer vision image targets that can be tracked in both optical and fluoroscopic X-ray images, ability to recalibrate the nail hole locations to address nail and deflection and deformation that may take place during the insertion process using fluoroscopic X-ray images obtained in the operating room as a normal part of the surgical procedure, and ability of the system to calibrate and use IMU orientation sensor data in place of orientation estimates obtained from optically-based methods to improve tracking performance and address issues of occlusion.

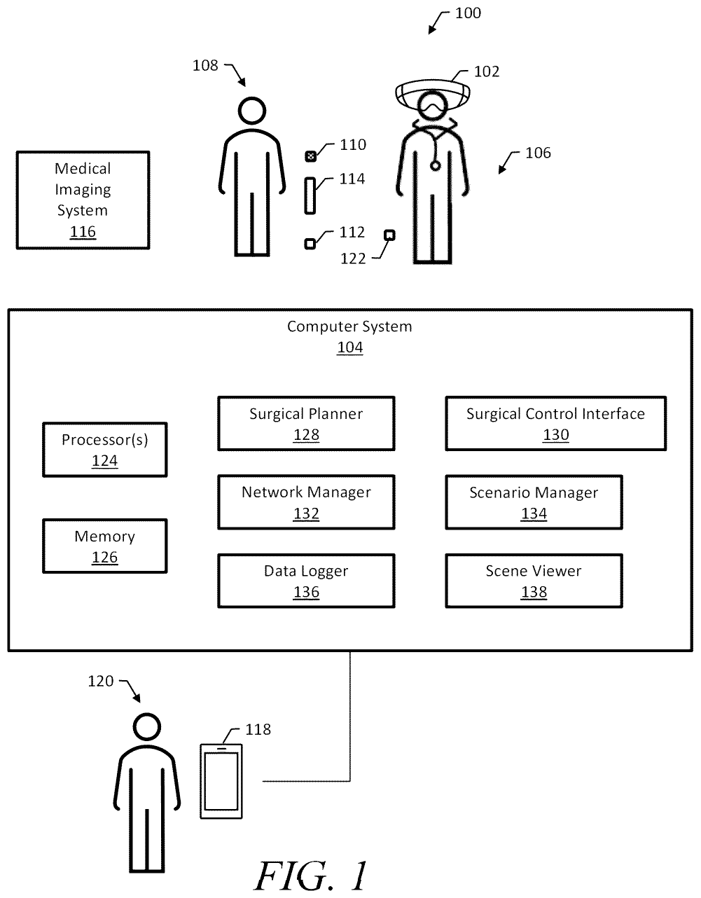

FIG. 1 is a block diagram of an example extended reality surgical environment 100 that includes an extended reality head-mountable display (HMD) 102 and a computer system 104 configured for providing surgical guidance to a surgeon 106 performing a surgical procedure on a subject 108. The computer system 104 is programmed for receiving images from the HMD and/or one or more external sensor devices (i.e. RGB, RGBD, infrared, Lidar, ultrasound, etc.) while the surgeon 106 is performing the surgical procedure and identifying at least one marker or marker-less image feature 110 depicted in the camera images.

The computer system 104 is programmed for tracking the position and orientation of at least one surgical object or device using the image feature 110; for example, the computer system 104 can be programmed for tracking a fixation device 112 and an anchor device 114. The computer system 104 is programmed for sending one or more visual indicators to the HMD 102 to display a target location for the surgical device to the surgeon for the surgical procedure. In some examples, the computer system 104 is programmed for receiving one or more additional images of the subject from a medical imaging system 116 external to the HMD 102 and recalibrating, using the additional images, the target location to address deflection or deformation or both of a surgically implanted device resulting from an insertion process.

In some examples, the computer system 104 is programmed for displaying one or more surgical scene images on one or more mobile devices 118 used by other participants 120 of the surgical procedure. The surgery state can be displayed. Then, the computer system can be programmed for receiving at least one annotation of the surgical scene images from one of the other participants 120 and sending the annotation to the HMD 102 to display to the surgeon 106 for the surgical procedure. The computer system 104 can be programmed so that tracking the location of at least one surgical device includes receiving and calibrating sensor data from at least one inertial measurement unit (IMU) orientation sensor 122 or other self-referencing orientation and/or position sensor devices.

The computer system 104 includes at least one processor 124 and memory 126 storing instructions for the processor 124. The computer system 104 can include one or more of: a surgical planning tool 128 a surgical control interface 130, a network manager 132, a scenario manager 134, a data logger 136, and a scene viewer 138. Surgical planning tool 128 is configured for executing a pre-operative planning mode and creating a customized surgical plan based on pre-operative subject image data. The data logger 136 is configured for data and video recording and logging to enable the surgical procedure to be replayed and/or viewed at a later point in time.

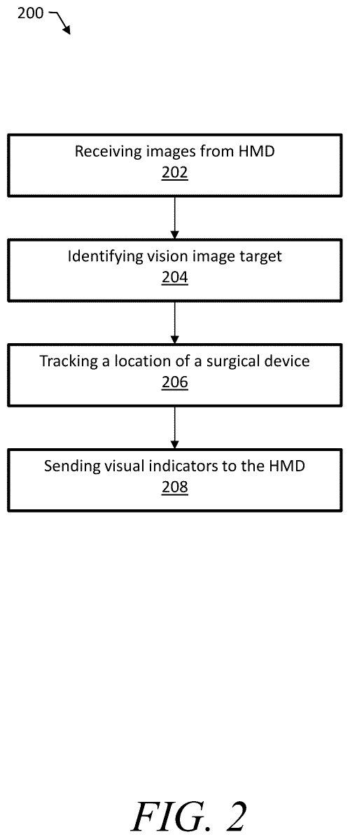

FIG. 2 is a flow diagram of an example method 200 for extended reality-based systems for surgical navigation. The method 200 can be performed by a computer system having one or more processors.

The method 200 includes receiving images from external cameras in the room and/or an extended reality head mounted display (HMD) while a surgeon is wearing the HMD and performing a surgical procedure on a subject (202). The images can be taken from one or more forward-facing cameras on the HMD that represent the surgeon's view.

The method 200 includes identifying at least one image feature depicted in the images (204). The image feature can be an image of a fiducial marker, for example, a 2D image target attached to the surgical device or other location relevant to the surgical procedure, the centroid of the surgical device or another 3D object that represents its position and orientation in the space, the surface of the surgical device or 3D object, bounding volume of the surgical device or 3D object, etc.

The method 200 includes tracking a location of at least one surgical device using the image feature (206). The method 200 includes sending one or more visual indicators to the HMD to display a target location for the surgical device to the surgeon for the surgical procedure (208).

### Computer Assisted Surgical Navigation

Minimally invasive surgical procedures continue to be popular due to their relative simplicity, lower cost, and reduced patient recovery times. Because such procedures generally restrict the surgeon's ability to see the operative area, surgeons generally rely on computer systems, such as computer assisted surgical navigation (CASN) systems to assist in the surgical operations.

### Augmented Reality

Augmented reality (AR) technologies have opened up new possibilities for improving the efficacy of surgical procedures through generation and display of real-time graphics (based on patient-specific medical data) that help guide and assist the surgeon during a procedure. This is especially relevant for many orthopedic surgical procedures and applications. For example, such a system can be used to provide a surgeon with “X-ray vision” by displaying 3D graphical models and other imagery using smart AR glasses (such as the Microsoft HoloLens) illustrating internal surgical anatomy normally hidden to the surgeon. This could also include, but is not limited to, highlighting pertinent anatomical landmarks and implant placement locations, including safe corridors for drill and screw trajectories associated with the surgical procedure being performed.

### Intramedullary Nailing Systems

Advanced orthopedic trauma care resorts to minimally invasive surgical procedures through intramedullary (IM) nailing, plating, or pinning (i.e., external skeletal fixators) of long bones utilizing stainless steel, titanium, titanium alloy or various material composites. When an intramedullary nail is used, the nail is typically inserted through a small soft tissue portal via a small soft tissue incision and a drill hole to open the medullary canal of the target bone (i.e., tibia, femur, humerus) using fluoroscopic guidance. Using closed fracture reduction techniques, the nail stabilizes the fractured bones by holding the bones in anatomic alignment. Locking screws are then placed through the proximal and distal ends of the nail, in a direction perpendicular to the long axis of the nail, in order to anchor the nail to the adjacent bone cortices. This provides angular and rotational stability to the construct.

Current intramedullary nailing systems feature a physical guide (i.e., insertion handle) for the insertion of the nail and an aiming arm attached to the insertion handle for the placement of the proximal screws. Increasing the length of the aiming arm to align with the distal screw holes is not practical due to decreased accuracy (especially with long nails in femur and tibia). As a result, increased surgeon skill is required for what is called a free-hand technique (“bulls-eye”) that drills holes using extensive amounts of radiation via intraoperative fluoroscopy to ensure successful placement of the distal interlocking screws. These challenges often result in decreased utilization of intramedullary nailing for long bone fractures despite its benefits of early rehabilitation and a more stable fixation of the fracture.

### Existing Intramedullary Nailing Systems

A number of different non-computer-based approaches have been taken over the years to address these challenges. External aiming arms (i.e., “jigs”) for distal screw insertion have been tried with limited success due to 1) inaccuracies in the mounting between the insertion handle and jig, and 2) misalignment errors between the actual distal hole locations in the nail and the corresponding hole locations in the jig resulting from deformation of the nail after it is inserted into the bone. Radiolucent drills and drill guides have also been developed to improve the speed and accuracy of hole placement, but still require a significant number of X-ray images to be taken to align the drill with the nail hole.

Computer assisted surgery systems supporting intramedullary nail insertion for the fixation of long bone fractures have been contemplated since the late 90s. Early systems, such as those described in Keinzle, U.S. Pat. No. 6,922,581, require three orthogonal fluoroscopic x-ray images to be captured, along with optical tracking of the insertion handle and drill guide to determine the location and orientation of a distal nail hole. In the Keinzle system, the position and orientation of the insertion handle and drill guide are tracked in space using locally attached LEDs, with an external tracking camera mounted on associated equipment in the operating room. Knowing the position and orientation (i.e., pose) of the drill guide and insertion handle allows associated graphical representations to be superimposed on captured intraoperative X-ray images. The estimated pose information of the insertion handle and drill guide are also used to estimate the current drill bore trajectory which appears as a graphical overlay on the three orthographic x-ray images shown to the surgeon on a display screen. In order to use this system, the surgeon is required to gaze away from the patient to view the orthographic x-ray views, then move the drill guide along the patient anatomy in order to line up the predicted drill trajectory with the distal nail hole location and axis in all three views. Once this is accomplished the surgeon is able to drill the hole.

The Trigen Sureshot Distal Targeting System (Smith & Nephew Inc.) utilizes electromagnetic (EM) tracking technology to project virtual images of the distal hole and the drill onto a display screen in order to enable the insertion of distal interlocking screws without X-ray fluoroscopy. The advantages of using EM tracking over optical tracking is that it has no line-of-sight (i.e., occlusion) problems and can accommodate issues associated with small changes in the distal nail holes due to deformation of the IM nail during the insertion process. However, electromagnetic sensors and systems suffer from tracking errors and inaccuracies caused by magnetic field distortion that results from proximity to ferromagnetic objects and materials and magnetic field producing devices such electric motors typically found in an operating room setting.

Ma et al. (2018) proposes a hybrid system that combines optical tracking of a drill [and/or drill guide] with electromagnetic tracking of a small receiver inserted into the IM nail to determine the position and orientation of distal IM nail holes. An electromagnetic sensor is inserted into the IM nail from the proximal end to the distal end, then fixed in place with respect to the nail. The main focus of the approach is to improve the tracking accuracy of the system in clinical situations by correcting for errors that occur due to 1) deformation of the nail during the insertion process and 2) for global errors that occur in the electromagnetic field due to electromagnetic interference. A fixed optical see through (OST) video display device positioned over the surgical site is used to display surgical navigation information to the surgeon, including changing the color of a graphical overlay representing the drill path to green, yellow, or red depending upon the magnitude of the position and orientation errors. Issues regarding placement of electromagnetic transmitter in the OR, especially with respect to proximity to the sensor embedded in the nail as well as sources of electromagnetic interference need to be addressed in such a system. It is also not clear what the FDA approval process is for embedding the electromagnetic sensor in the nail as part of the surgical procedure.

Similar to Ma et al. described above, Tu et al. (2020) also describes a hybrid surgical navigation system using optical and electromagnetic sensors and devices. Like in Ma et al., the system uses tracking of optical targets to determine the correspondence between world and electromagnetic coordinates systems and to correct for electromagnetic field distortions. In addition, Tu tracks the position and orientation of the drill and a hole calibration pin using electromagnetic sensors and uses a Microsoft Hololens2 head-mounted display (HMD) as the optical see through (OST) display device. The system tracks the tip of the drill bit. A networked desktop application is also described that allows the electromagnetic sensor data to be sent to an application running on the Hololens that displays a graphical ring object representing the distance and angle deviations between the drill bit position and the desired drill path. The ring is rendered along the axis of the distal hole. As the distance and angle deviation errors change, the horizontal and vertical dimensions of the ring change proportionally. The ring turns green when both the distance and angle deviations are below defined threshold values.

Orthopractis has developed a handheld augmented reality iPhone app called “IMNailScrewAR” that allows the user to insert screws (drill bits) into distal nail hole locations by allowing the user to view the distance of the tip of a tool from three orthogonal planes (i.e., sagittal, coronal and transverse) associated with the nail in the real world. The desired distal nail hole locations and orthogonal planes are determined as part of a calibration phase by having the user physically place the tip of a probe (attached to the back of the iPhone) on both sides of two distal hole locations in the nail (i.e., front and back of holes). iPhone tracking of an image target attached to the insertion handle is used during the calibration phase to determine the position of the distal nail hole locations with respect to the image target as well as the sagittal, coronal, and transverse reference planes. During the screw placement phase the insertion handle image target is also tracked in real-time by the iPhone to determine the position of the tip of a tool (e.g., screwdriver) attached to the iPhone case probe with respect to the distal nail hole locations. A circular 2D dart board-like graphic composed of concentric circles divided by lines (which they call a “radar” screen) appearing on the iphone display screen provides guidance to the user during the screw placement process by displaying a dot that represents the location of the tip of the tool with respect to the desired nail hole location. The system also overlays 3D rectangles representing the sagittal, coronal, and transverse planes on top of the nail at their corresponding physical locations in the real world. As the user moves the handle of the tool (i.e., screwdriver) the size of the dot and its location on the radar screen changes in real time. The dot size is proportional to the inverse of the distance of the tool tip to the distal nail hole location. That is, the closer the tool tip to the hole location the bigger the size of the dot on the radar screen. The quadrants of the radar screen (left up, left down, right up, right down) also enable the user to view the tool tip location with respect to the coronal and transverse orthogonal planes. The iPhone display screen also displays the current distance to the hole location in each of the planes in mm and the angle of the tool to each of the planes in degrees. According to the authors, the IMNailScrewAR iPhone App is targeted at orthopedic surgery education and training applications and is not intended to be used in actual surgical procedures. As an iPhone-based app, the user needs to hold the phone in one hand during usage. In addition, it appears that the system only supports the use of a screwdriver tool that connects to the iPhone through a mount attached to the end of the iPhone probe. As a result, it is not clear from the descriptions or videos provided if the system can be used to drill distal nail holes (especially as part of an actual surgical procedure) since a user typical holds a soft tissue protector (i.e., drill guide) in one hand and the drill in the other hand, leaving no hands free to hold and position/orient the iPhone.

7D Surgical has developed an image-guided surgical system that allows volumetric data obtained from CT and MRI scans to be quickly registered with associated patient anatomy.

### Unmet Need

Although there has been progress to date on surgical guidance systems supporting intramedullary nail insertion, there currently is still an unmet clinical need for determining drill hole locations for distal interlocking screws as well as robust surgical guidance (for example, not prone to errors introduced by electromagnetic interference) for inserting such distal screws in IM nailing systems used in typical operating room environments. Key problems include:
error prone distal nail hole targeting systems and devices;
the need for increased technical skill for surgeons desiring to use a free-hand drilling technique;
increased radiation exposure to the surgeon and patient during free-hand techniques; and
increased operating time.
   As a result of this unmet need, there has been decreased use of IM nailing systems.

### Advantages

The surgical navigation described herein has a number of features that distinguish it over existing systems. These include: 1) the use of self-referencing s systems and sensors, such as head-mounted computer vision systems and wireless inertial measurement unit (IMU) orientation sensors (i.e., eliminating need to set up an external optical camera or electromagnetic sensor systems in the operating room), 2) the use of computer vision image targets that can be tracked in both optical and fluoroscopic X-ray images, 3) the ability to recalibrate the nail hole locations to address nail deflection and deformation that takes place during the insertion process using fluoroscopic X-ray images obtained in the operating room as a normal part of the surgical procedure, 4) the ability for other participants using mobile devices, such as phones and pen-based tablets to markup and annotate the surgical scene using the touchscreen of the mobile device, and 5) the ability of the system to calibrate and use IMU orientation sensor data in place of orientation estimates obtained from optically-based methods to improve tracking performance and address issues of occlusion.

### Technical Description

The extended reality surgical navigation system described herein addresses the unmet need by providing real-time intraoperative guidance when placing locking screws into the distal end of IM nails without the need for concurrent fluoroscopic guidance or for accurate screw placement during minimally invasive plate osteosynthesis. The AR approach taken is compatible with existing implant systems, making the surgical navigation system described herein agnostic to use with commercially available intramedullary nailing solutions, thereby enhancing its utility and commercial versatility.

### Typical Nail Insertion Procedure

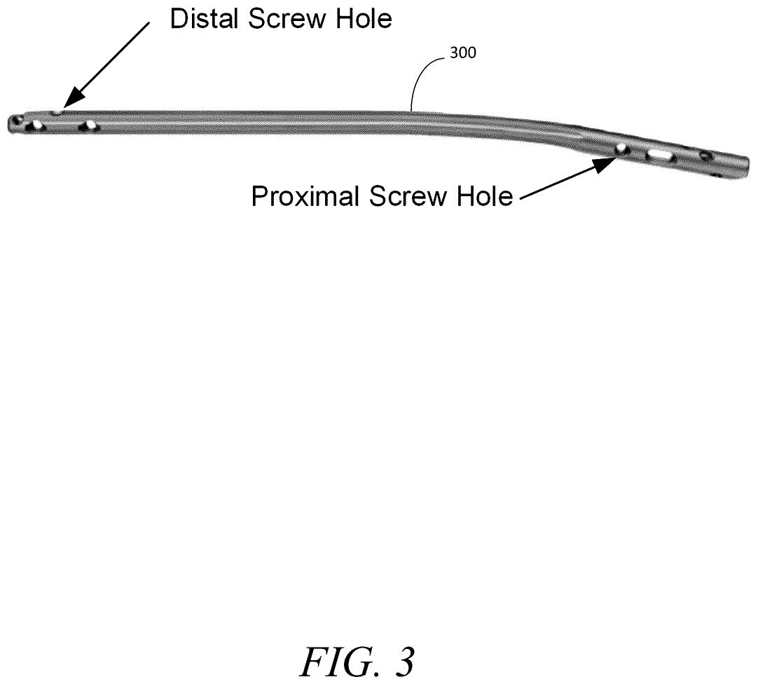

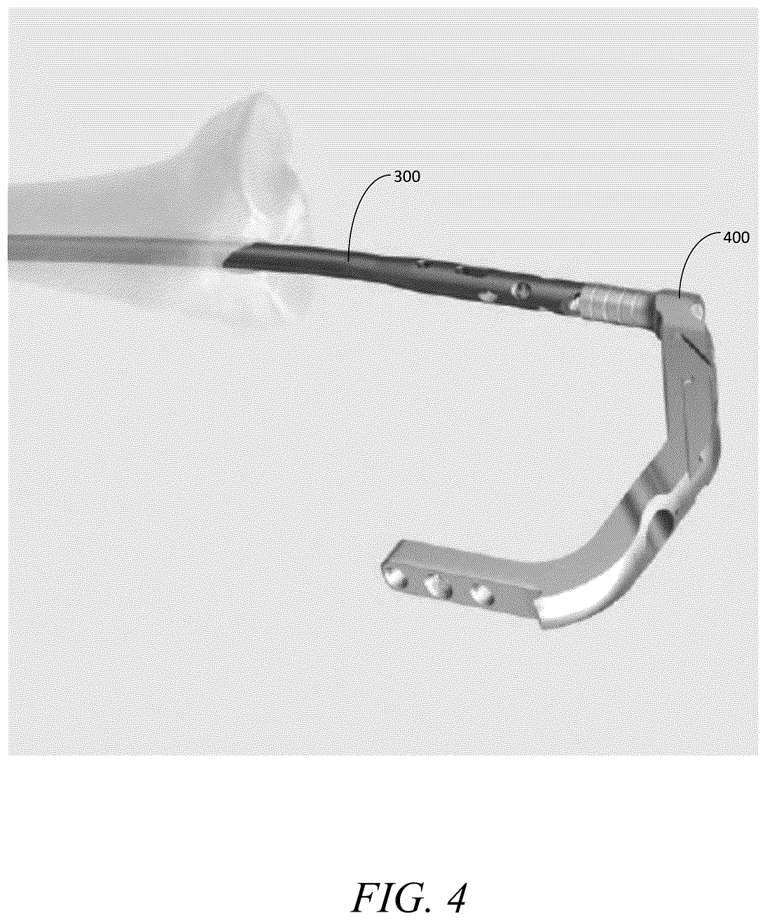

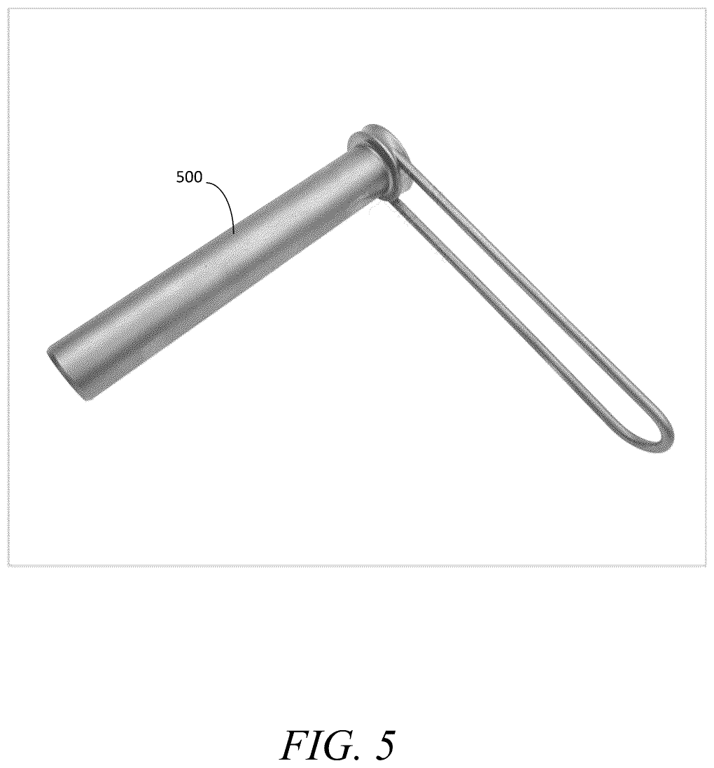

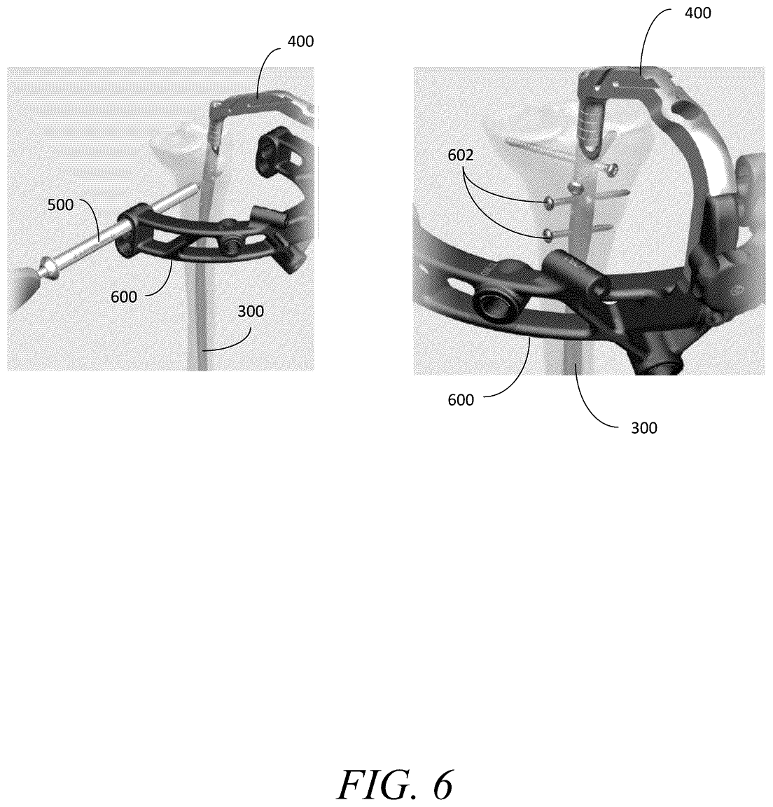

A typical tibial nail, insertion handle and soft tissue protector (i.e., drill guide) are shown in FIGS. 3-5. To insert a tibial or intramedullary (IM) nail 300 (FIG. 3) in the IM canal an insertion handle 400 (FIG. 4) is rigidly attached to IM nail 300. To place the proximal locking screws, an aiming arm 600 (FIG. 6) is mounted onto insertion handle 400 attached to intramedullary nail 300 (FIG. 3). The sidebar of aiming arm 600 allows for targeting and installing the proximal locking screws 602 through the near side of bone, across the nail and out the far side of the bone in FIG. 6. The distal locking screws are installed through the distal screw holes (FIG. 3) on the end of intramedullary nail 300 opposite the end where insertion handle 400 and aiming arm 600 are attached to intramedullary nail 300. A drill guide 500 illustrated in FIG. 5 is used to set the position and orientation of a drill so that the surgeon can drill holes through the bone that align with the proximal and distal screw holes in intramedullary nail 300. Drilling the holes through the bone to pass through the proximal screw holes in intramedullary nail 300 is less error prone because of the fixed relationship between there is little flexure of aiming arm 600 at the proximal end of the bone. However, as will be described in more detail below, at the distal end of the bone, there can be flexure and rotation of the aiming arm relative to the intramedullary nail, which can cause the drilling location to be off center in the bone and not aligned with the screw holes in intramedullary nail 300

Intraoperative fluoroscopy is often required for targeting the location of the distal locking screws which is then accomplished using either a free-hand technique or a variety of radiolucent supportive devices for improved accuracy. Currently available methods for effectively targeting the location of distal locking screws for IM nails are technically problematic and result in increased intraoperative exposure to ionizing radiation (fluoroscopy) and significantly dampen surgeon enthusiasm for intramedullary nailing systems.

### Extended Reality Surgical Navigation System

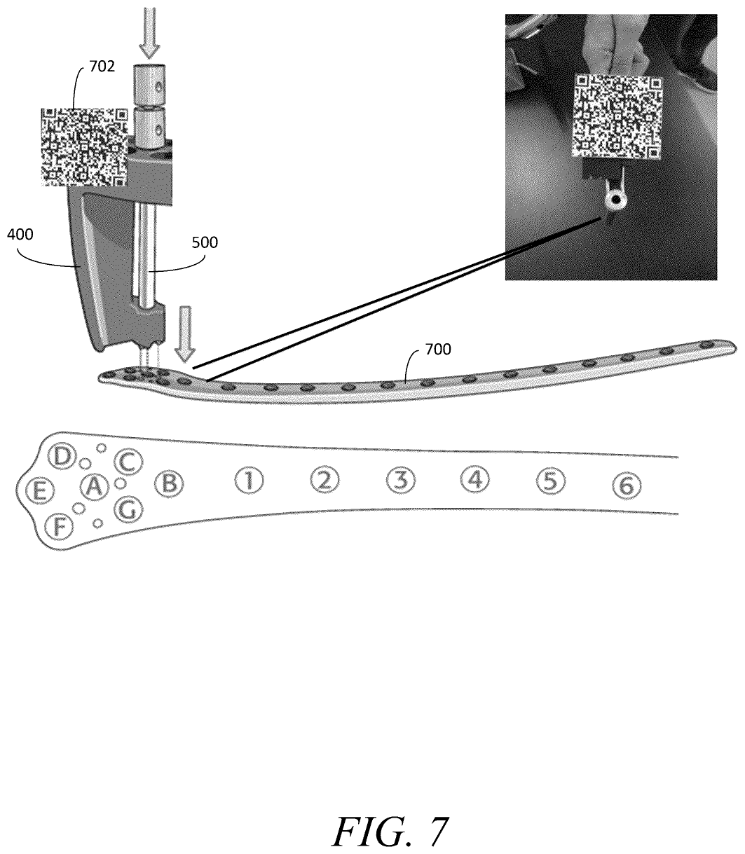

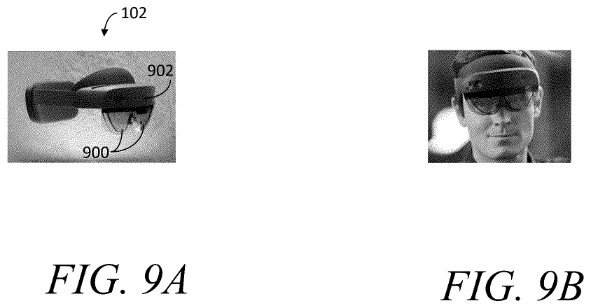

The extended reality surgical navigation system described herein enhances and extends existing intramedullary nailing systems by providing a platform for both preoperative planning of the procedure and intraoperative guidance of implant placement. It is also expandable beyond the application of intramedullary nails. Although intramedullary nailing is the ideal approach, this method is not free of complication either and is not applicable where the fracture is adjacent to joints or when there is intra-articular component with extension to the metaphyseal-diaphyseal junction. This method would not be ideal in circumstances, such as: diaphysis fractures extended to the metaphysis or joint, children having epiphyseal growth plates. MIPO or biological plating and bridging plate has recently become more popular but its outcomes for comminuted fractures have not been thoroughly investigated. The bridging plate method is named due to the action of the plate as a bridge between the proximal and distal bones. This provides stability while preserving the vasculature integrity of the soft tissue. MIPO technique using a locking compression plate (LCP) 700 (see FIG. 7) has been used widely in trauma cases. Its advantages are that the MIPO technique does not interfere with the fracture site and thus provides improved biological healing, and that LCP 700 has excellent angular stability. After adequate reduction is obtained, the bone plate is inserted over the fractured bone and under the soft tissue envelope using insertion guide (FIG. 7). LCP 700 is no longer visible to the surgeon akin to the tibial nail, once introduced into the tibia. Using the same methodology as described for the tibial nail, a smart insertion handle 400 and a smart drill guide 500 can be used to guide the surgeon to place the bone screws through small percutaneous stab incisions using augmented navigational support. Insertion handle 400 and drill guide 500 are referred to herein as “smart” because the position and orientation of drill guide are tracked and used to display a virtual drill axis to the surgeon via head-mountable display 102. In FIG. 7, the position and orientation of drill guide 500 is tracked using a marker 702 attached to insertion handle 400. In the illustrated example, marker 702 comprises planar member attached to insertion handle 400 and oriented such that the surface containing the two-dimensional bar code is perpendicular to the drilling axis. The location and orientation of marker 702 may be tracked using one or more sensors, such as a visual light camera (see FIG. 9A) and used to display a virtual drilling axis to the surgeon.

The subject matter described herein is not limited to using a marker 702 to determine the position and orientation of drill guide 500. In an alternate implementation, desktop application 802 or an application running on the hardware of head-mountable display 102 may utilize computer vision or machine learning techniques to recognize drill guide 500 in video frames and track the location and orientation of drill guide 500 relative to a target drilling location and orientation.

The same concept of surgical navigation using the components of the extended reality surgical navigation system described herein can be expanded to other trauma care procedures using existing hardware components, such as, but not limited to pelvic reconstruction, corrective spinal procedures, arthroplasty, craniomaxillofacial and dental.

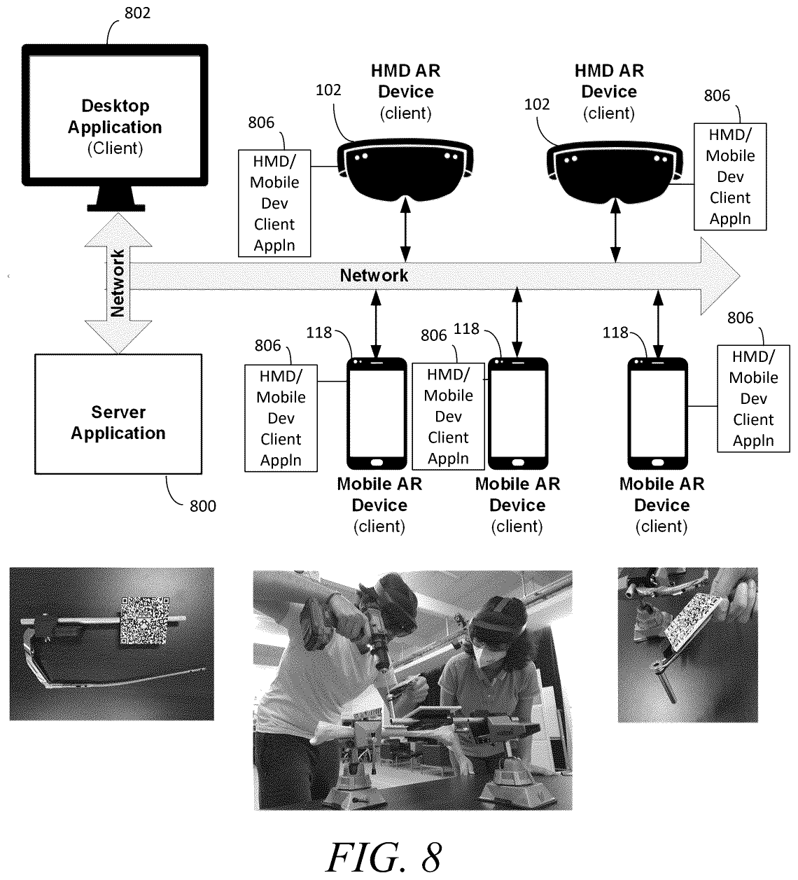

As shown in FIG. 8, the extended reality surgical navigation system consists primarily of a server application 800, a desktop application 802, and multiple extended reality viewer devices.

In FIG. 8, desktop application 802 creates a surgical plan and instructions, logging data associated with the surgical procedure and controlling the execution of the surgical plan appearing in the surgeon's HMD 102. Surgical instructions include a sequence of steps to be performed by the surgeon along with a description of what to do in each step. An example of such surgical steps is described in detail below.

Server application 800 may run either on a desktop machine or on a platform capable of communicating with desktop application 802 over a local area network (LAN). Server application 800 communicates the surgical plan, smart device sensor data and desktop application commands to/from HMD/mobile device client applications 806 executing on networked HMDs 102 and mobile AR devices 118.

FIGS. 9A and 9B respectively illustrate a head-mountable display suitable for use with the extended reality surgical navigation system described herein and a user wearing the head-mountable display. In FIG. 9A, head-mountable display 102 includes a visor-like form factor with displays lenses configured to be located in front of each of the users. Head-mountable display includes video see through holographic display lenses 900 that allow the user to view the real world and that allow holographic images to be projected onto their respective viewing surfaces so that the user can view holographic images overlaid on the real-world scene. Other HMD display embodiments are possible, including, but not limited to, pass-through video displays, direct retinal imaging displays, etc. Head-mountable display 102 includes a visual light camera 902 that captures video images of the real world from the wearer's point of view. Such video may be used to create the display shown on mobile devices 118 so that other participants can view and/or assist with a surgical procedure. Other components of head-mountable display 102 that are not visible in FIGS. 9A and 9B include position sensors, which sense the position and orientation of the wearer's head, a depth sensor, which measures depth in the real world scene, speakers, and microphones.

In FIG. 9A and 9B, the Microsoft Hololens is shown as one example of a head-mountable display. However, the subject matter described herein is not limited to using the Microsoft Hololens display. Any suitable head-mountable display with the capability of presenting mixed reality views to the user is intended to be within the scope of the subject matter described herein.

Head-mountable display 102 uses surgical plan data obtained from the desktop application 802 to generate and display text-based instructions, graphical markups, and annotations, as well as 3D computer-generated objects that appear to be physically located (i.e., embedded) in the real world. Examples of such objects, markups, and annotations include locations of distal nail holes, surgical guides, and markers (such as lines, circles, cylinders, cones, and other 3D geometry that assist the surgeon in drilling holes in the proper location that are also aligned with an axis perpendicular to the targeted distal hole location. These instructions, graphical annotations and markups are displayed to the surgeon via head-mountable display 102 to provide real-time interactive guidance during the surgical procedure. Additional head-mountable display devices may also be supported to enable teams of surgeons to jointly participate in complex surgical procedures through a spectator mode. In the spectator mode, desktop application 802 may display, on the holographic display lenses of the head-mountable displays worn by the spectators, video from the visual light camera 902 of the head-mountable display worn by the surgeon, including the virtual surgical guidance objects and any annotations.

Standalone head-mountable display devices, such as the Microsoft HoloLens, are wearable computers with see-through head-mounted displays allowing users to interact (through gaze, voice, and hand gestures) with 3D models (sometime called “holograms”), graphical models and other information that are superimposed (i.e., overlaid) onto the real-world environment. In the proposed AR surgical navigation system, head-mountable display 102 may be sterilized and put on after the surgeon has entered the sterile field.

Mobile devices 118 may be handheld mobile devices, such as Android and iPhone smartphones and tablets, that include augmented reality software (i.e., mobile AR viewers), that enable others in the operating room (medical students, nurses, technicians) to view the augmented reality content displayed to the surgeon from their own perspective through a Spectator Mode (that also considers their physical position and orientation in the room). Mobile AR viewer devices also include functionality enabling users to virtually “markup” the surgical area with hand-drawn lines, text, and other annotations, using the touch screen of the mobile device or alternatively a pen-based interface, to provide additional instructions, guidance, and direction to the surgeon.

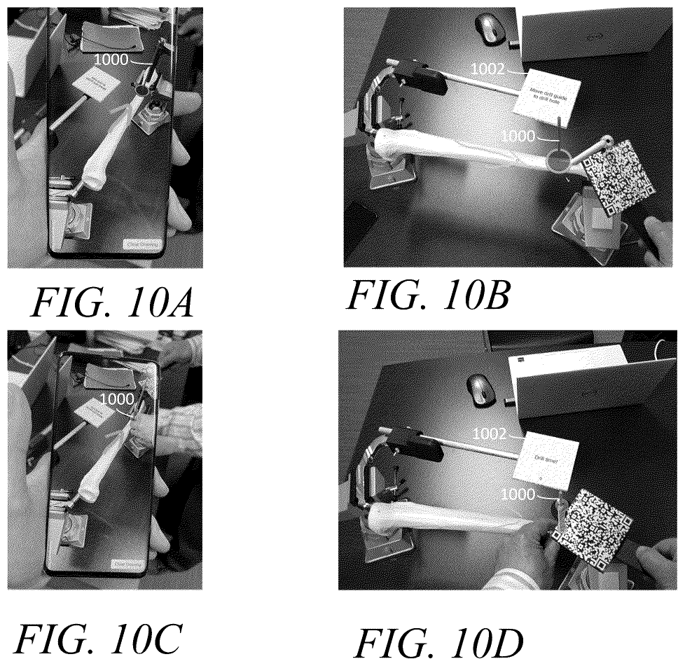

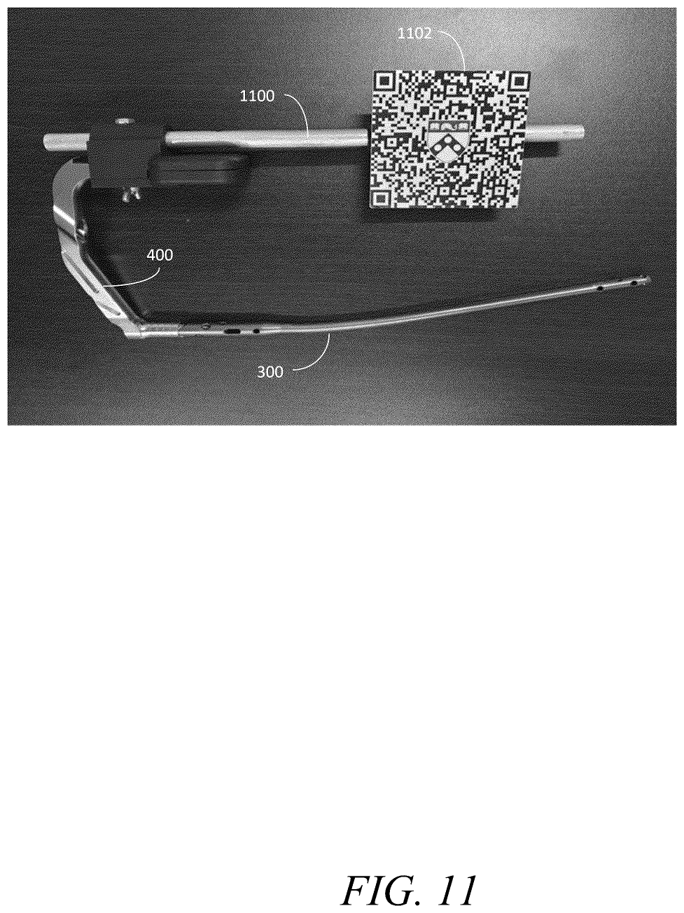

FIG. 10A shows the display of a mobile device with an annotation (an arrow in this case) added by the mobile device user instructing the extended reality headset wearer to move the drill guide in the direction of the arrow. In FIG. 10A, the arrow indicates that the extended reality headset user should move the base of the drill guide to the left to reduce position error. The green arrow shown on the bone was drawn using touchscreen of the mobile device. Such markups appear fixed (i.e., stationary) in the 3D physical world, regardless of the mobile device user or extended reality headset user point of view. FIG. 10B illustrates the corresponding view shown on the display of the extended reality headset.

FIG. 10C illustrates the display on the mobile device after the extended reality headset wearer has moved the drill guide to the target location. In FIG. 10C, it can be seen that when the drill guide is located at the target location and oriented correctly with the displayed virtual drill axis, the displayed virtual drill axis changes color from red to green. FIG. 10D illustrates the corresponding view shown on the display of the extended reality headset. FIGS. 10A-10D also illustrate the display of textual instructions 1002 to the surgeon instructing the surgeon as to how to position and orient drill guide 500.

FIG. 11 is a perspective view of a smart insertion handle assembly. The surgeon inserts nail 300 into the medullary canal using an intramedullary nail rigidly attached to insertion handle 400. The smart insertion handle allows the system to determine the position and orientation of the distal screw holes of the nail. The smart insertion handle includes a sensor mount positioned on extension arm 1100 and which can support a wireless IMU orientation sensor, infrared emitter/detector, and/or fiducial markers (i.e., “image targets”) which can appear in both optical and fluoroscopic X-ray images. In FIG. 11, marker 1102 is mounted on extension arm 1100.

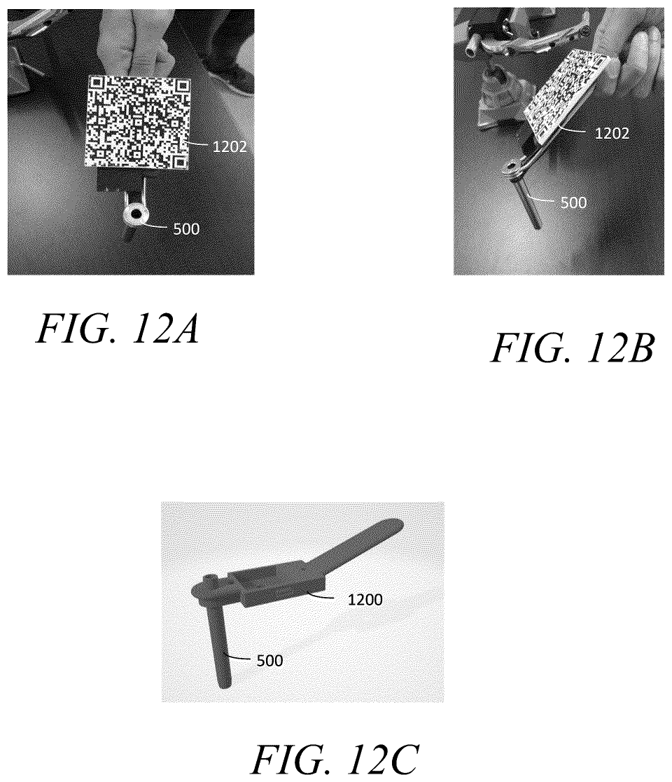

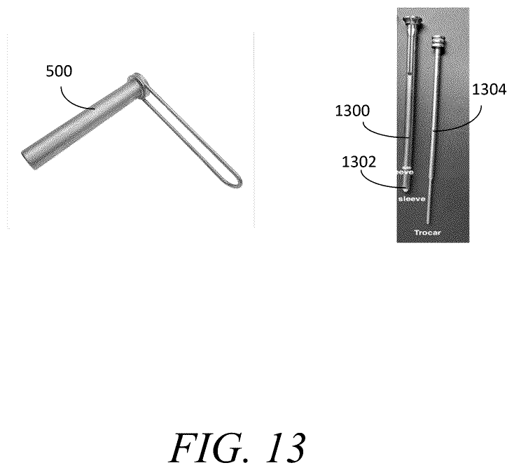

FIGS. 12A-12C are perspective views of a smart drill guide assembly. FIG. 13 illustrates exemplary components of a drill guide. In FIGS. 12A-12C, the smart drill guide assembly includes drill guide 500, a sensor mount 1200, and a marker 1202. The smart drill guide assembly (i.e., soft tissue protector) shown in FIG. 12A allows the surgeon to drill screw holes at the proper location with the correct orientation. Sensor mount 1200 can support rt a wireless IMU orientation sensor, infrared emitter/detector, and/or fiducial markers (i.e., “image targets”) which can appear in both optical and fluoroscopic X-ray images. As illustrated in FIG. 13, drill guide 500 includes an outer sleeve 1300, an inner sleeve 1302 that fits into outer sleeve 1300 (allowing for concentric placement of a drill bit) and a trocar 1304. Trocar 1304 is a pointed plunger that fits into inner sleeve 1302 that allows for placement and centering of the drill sleeve assembly prior to drilling. To address sterilization issues associated with the drill guide sensor mount and image targets, a disposable plastic drill guide (for example, 3D printed) shown in FIG. 12C, can also be used.

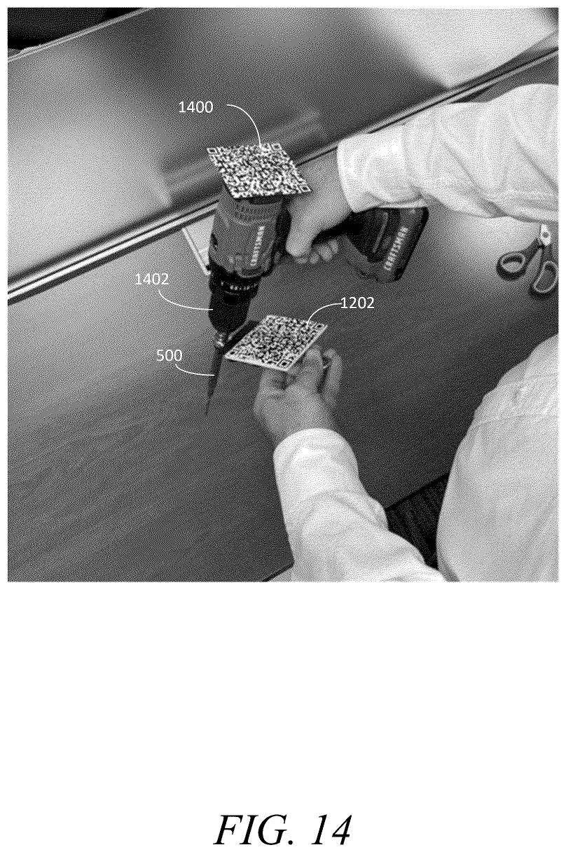

FIG. 14 is a perspective view of a smart drill guide with an additional sensor for determining drilling depth. In FIG. 14, the smart drill guide assembly includes drill guide 500, sensor mount 1200, and marker 1202. The smart drill guide may also include an optional depth sensor (infrared, magnetic, optical, etc.) (not shown in FIG. 14) mounted at the top of the drill guide that measures the distance between the sensor and a reflective collar attached to the drill bit. This depth sensor provides real-time data enabling the surgeon to drill a screw hole not only with the correct position and orientation, but also with a depth appropriate for the length of the screw. As an alternative, an image target, such as marker 1400, can be attached to the top of drill 1402 and used to determine the depth of the drill hole at any point in time based on the difference between the relative position of marker 1202 attached to drill guide 500 and marker 1400 attached to drill 1402 at the start of the drilling and any other instance in time.

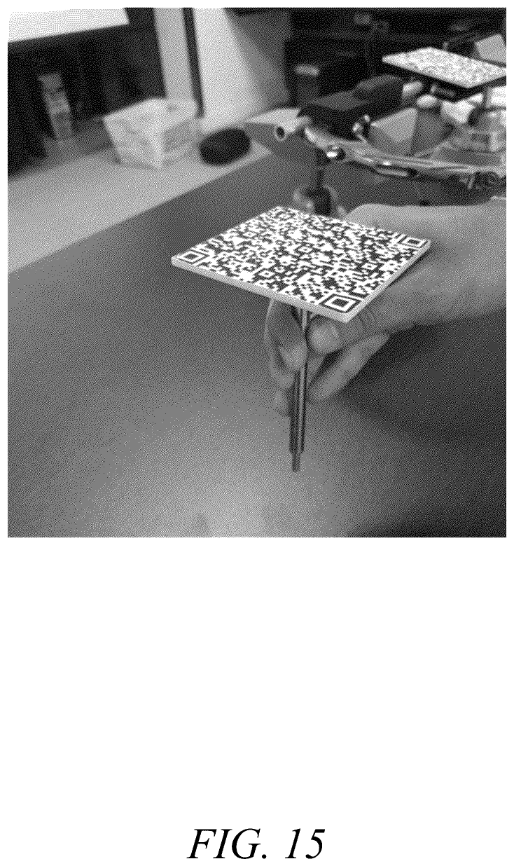

FIG. 15 is a perspective view of a smart calibration pin suitable for calibrating the surgical navigation system. Referring to FIG. 15, a smart calibration pin 1500 includes a fiducial image target, such as marker 1502, that allows the system to determine the position and orientation of the distal screw holes with respect to the image target attached to the insertion handle 400. As a result, calibration of the system can be completed in a matter of seconds and does not require any prior knowledge of the nail type or model or its technical specifications, including use or creation of 3D models or CAD models.

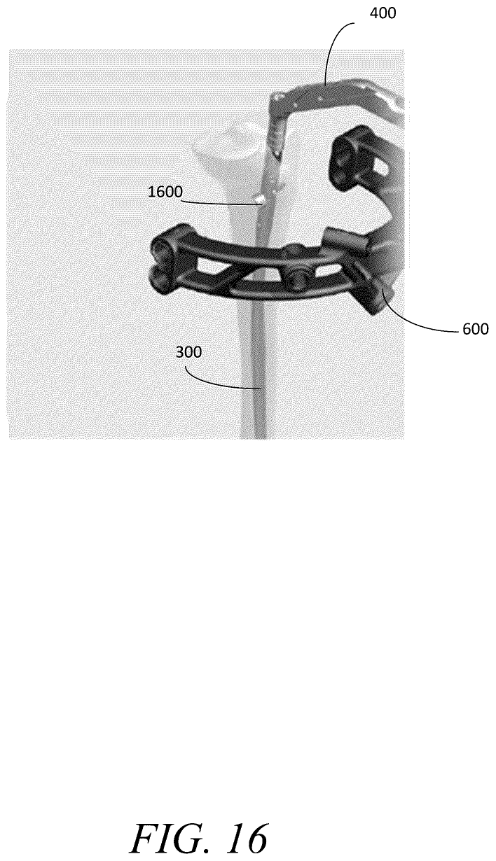

In addition, nail orientation data can be obtained from a wireless inertial sensor packaged as a smart screw which is inserted into the proximal holes of the nail. FIG. 16 illustrates an example of a smart screw. In FIG. 16, a smart screw 1600 is inserted into one of the proximal screw holes in intramedullary nail 300. The wireless inertial sensor contained within smart screw 1600 can also provide patient orientation and acceleration data post-operative for tracking rehabilitation, recovery, and patient outcome.

As shown in FIG. 8, the surgical navigation system includes three main software components: desktop application 802, server application 800 and HMD/mobile device client applications 806.

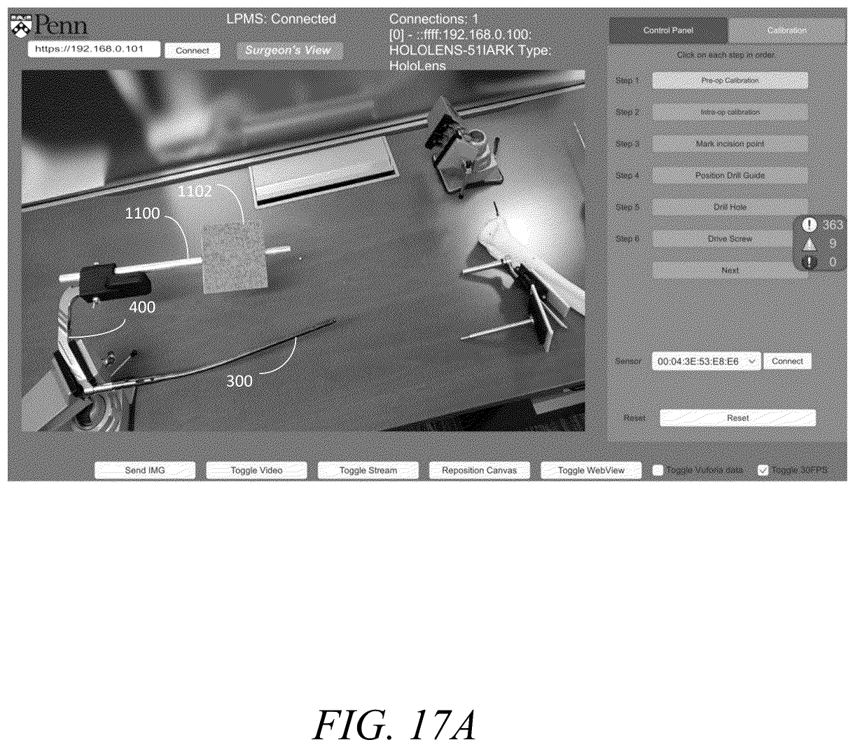

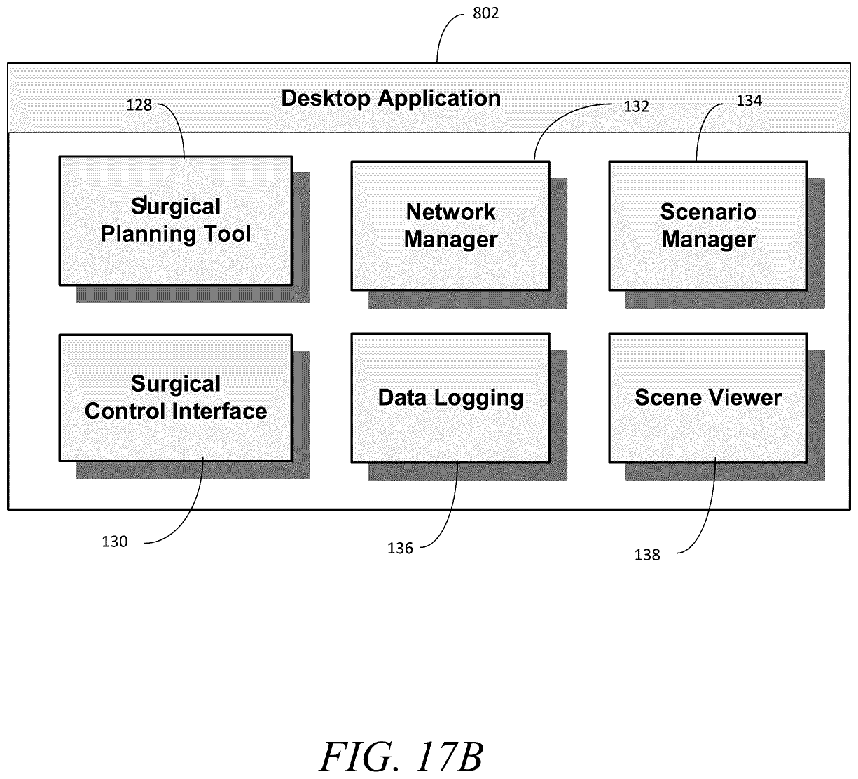

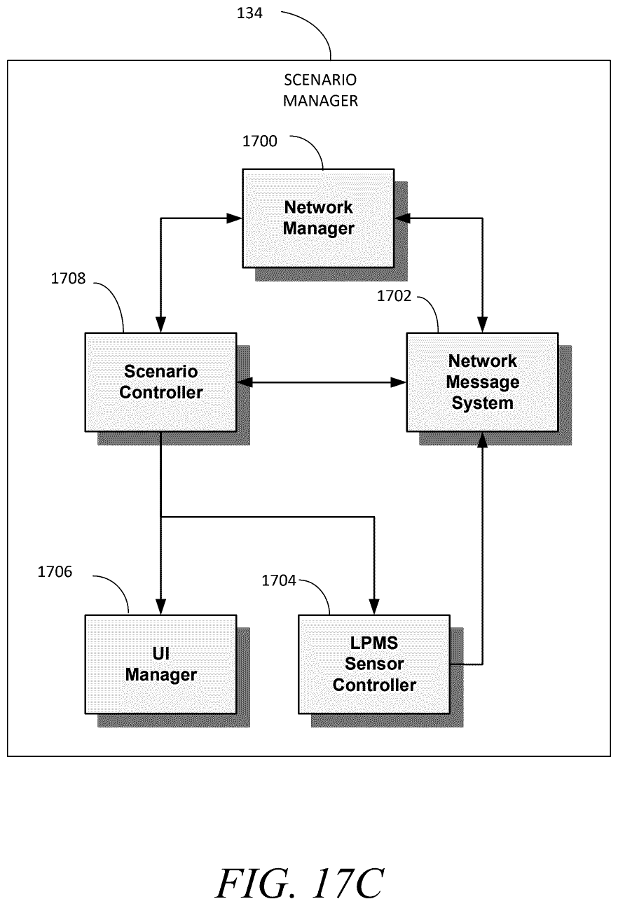

FIG. 17A illustrates an example of a user interface that may be displayed by desktop application 802. In FIG. 17A, the user interface includes a video display from the perspective of visual light camera 902 of HMD 102 worn by the surgeon. In the illustrated example, the surgeon's view shows intramedullary nail 300, insertion handle 400, sensor mount 1000, and marker 1102. The user interface also illustrates a control panel that includes a series of steps for the surgeon to perform in drilling the holes and inserting the screws. The user interface further includes a calibration panel, which is not shown in FIG. 17A.

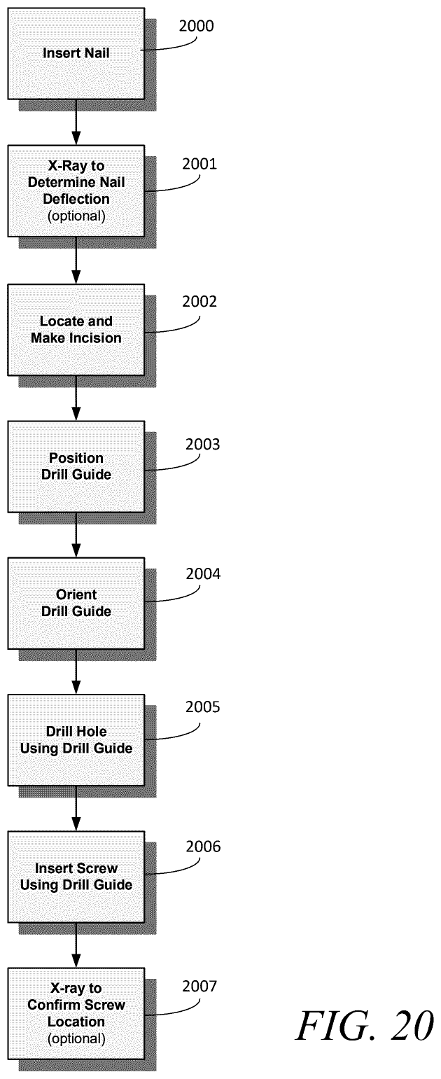

FIG. 17B illustrates exemplary components of desktop application 802. In the illustrated example, the components include surgical planning tool 128, surgical control interface 130, network manager 132, scenario manager 134, data logger 136, and scene viewer 138 described above with respect to FIG. 1. During the injury diagnostic process an imaging modality typically will be used (for example, computed tomography (CT)) and the CT image data can be used to generate a 3D model of the bone. This 3D bone model may be used by surgical planning tool 128 to begin the preoperative planning phase. Surgical areas of interest displayed by desktop application 802 can be viewed and examined to evaluate the extent of the injury (i.e., fractured bone). Using surgical planning tool 128, the surgeon can measure geometric dimensions and angles to develop an associated surgical plan. During the preoperative review of a patient's imaging data, a surgeon may mark three points surrounding each of a targeted screw location in order to define a 2-D plane and associated hole axis that enables appropriate graphical annotations to be displayed (such as those shown in FIGS. 20-25) that guide the surgeon during the hole drilling and screw driving procedures. Surgical planning tool 128 also can be used to identify safe anatomic corridors for drilling trajectories, implant location and placement, the direction of drill hole and locking screw trajectories for an intramedullary nail, the safe drill trajectory for a pedicle screw or the safe drilling trajectory for a bone plating procedure. Alternatively, an implant system and its technical specifications may be selected from a library (i.e., database maintained on the server) containing technical specifications of existing commercially available implant systems (i.e., intramedullary nailing equipment across several manufacturers) in order to configure a surgical plan. Custom or personalized patient implant designs can also be added to the database given appropriate technical specification and 3D CAD model data. The intent is to enable the library of implant systems accessible by surgical planning tool 128 to be kept current with new and evolving implant system designs. Following configuration and completion of the surgical plan, the plan's data, and instructions (and optionally with selected implant system model data), are saved locally and exported to the server application 800 for deployment to the desktop application 802 and HMD 102 in order to provide real-time guidance to the surgeon.

Surgical control interface 130 allows a user of desktop application 802 to control and sequence the information and graphics displayed to the surgeon in the HMD 102. For example, to remotely sequence the graphical models, annotations guiding the surgeon in each of the steps of a surgical procedure (such as the tibial nail insertion tasks illustrated in FIGS. 3, 5 and 19), to open image data files and other relevant patient data for display to the surgeon in HMD 102, to record and replay video captured by HMD 102, etc.

Network manager 132 is responsible for connecting and managing the communication of data among server application 800, desktop application 802, and HMD/mobile device client applications 806. Scene viewer 138 gives the user of the desktop application the ability to view the surgical procedure in real-time from both the surgeon's first-person perspective and also from a third person point of view. Video data representing what the user sees in the Hololens is streamed in real-time to the Desktop application over a local network connection. Data logger 136 gives the user of desktop application 802 the ability to record and replay the surgical procedure as well as save and load relevant surgical procedure data to/from a file. FIG. 17C is a block diagram illustrating exemplary components of scenario manager 134. In FIG. 17C, scenario manager 134 includes a network manager 1700, a network message system 1702, and LPMS sensor controller 1704, a UI manager 1706, and a scenario controller 1708. Network manager 1700 and network message system 1702 handle internal communications among components of scenario manager 134 and with other components of desktop application 802. Scenario manager 134 is responsible for communicating commands and data from surgical control interface 130 to HMDs 102 and mobile devices 118 as well as managing and controlling the display of graphical annotations, 3D models and other information appearing in the HMDs 102 and mobile devices 118. UI manager 1706 is responsible for controlling the various displays. LPMS sensor control 1704 controls an IMU that senses orientation of drill guide 500. Scenario controller 1708 controls the display of annotations and virtual objects by desktop application 802.

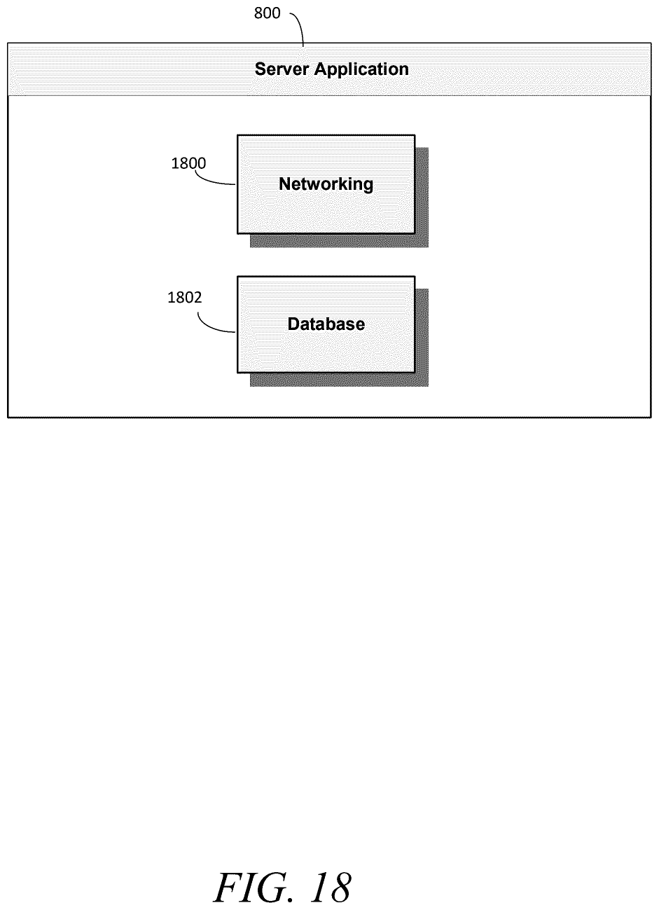

FIG. 18 is a block diagram illustrating exemplary components of server application 800. Server application 800 includes a network component 1800 is responsible for establishing and managing connections between the mobile, desktop and HMD AR device clients, sharing surgical procedure data (such as the position and orientation of the insertion handle and drill, depth of drill hole, etc.) between these clients and implementing a message handler that allows the surgical control interface to control the display of information and execution of software running on the HMD and mobile AR Viewer devices. The networking feature of the server application 800 also allows mobile device and additional HMD AR viewer users connected to the system to view the graphical models, annotations and other information displayed to the surgeon from their individual points of view. For example, to see the expanding and contracting cone used to orient the drill guide or to provide “X-ray vision” that allows them to see the position and orientation of the nail inserted in the patient's bone. The ability for users to also see what the surgeon sees, but from their own unique perspective, has both education and training applications.

Database 1802 maintained by server application 800 includes patient image, data such as CT or MRI scans, fiducial marker data such as insertion handle, drill guide and calibration pin image targets, 3D tracking data representing the position and orientation of tracked objects (such as the insertion handle, drill guide, drill bit, etc.) as a function of time, video recordings of surgical procedures (from the perspective of HMD 102 worn by the surgeon), as well as libraries of implant system information and technical specifications.

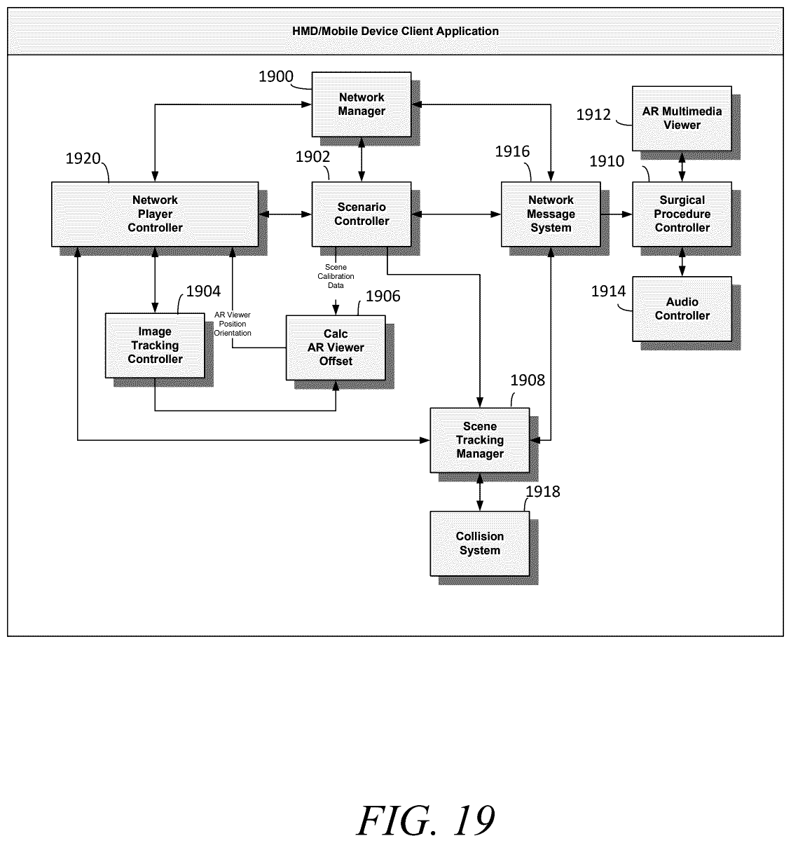

FIG. 19 is a block diagram illustrating exemplary components of HMD/mobile device client application 806. In FIG. 19, HMD/mobile device client application 806 includes a network manager 1900, a scenario controller 1902, an image tracking controller 1904, an A/R offset calculator 1906, a scene tracking manager 1908, and a surgical procedure controller 1910. Network manager 1900 is responsible for connecting and managing the communication of data between AR Viewer Devices and the server application 800. Scenario controller 1902 is responsible for receiving commands and data from server application 800, as well as managing and controlling the display of graphical annotations, 3D models and other information appearing in the AR Viewer devices. Image tracking controller 1904 Is responsible for tracking the image targets in the scene. AR offset calculator 1906 is configured to, given the position and orientation of secondary HMD AR or mobile AR viewer device points of view, compute position and orientation offset values so that when applied to 3D graphical objects received from server application 800 the 3D graphical objects appear properly positioned and oriented in the scene. Scene tracking manager 1908 allows mobile devices and HMD AR viewer users connected to the system to view the graphical models, annotations and other information displayed to the surgeon from their individual points of view. Surgical procedure controller 1910 provides the ability for the desktop user to control and sequence the information and graphics displayed to the surgeon in the HMD AR viewer.

HMD/mobile device client application 806 further includes an AR multimedia viewer 1912, an audio controller 1914, a network message system 1916, a collision system 1918, and a network player controller 1920. AR multimedia viewer 1912 provides the ability for a user to retrieve and view 2D images, 3D models and videos in the AR environment. As a result, users such as surgeons can easily place relevant patient specific data (for example, X-ray images, 3D CT or MRI models, etc.) at desired locations in the operating room or register such 3D models with respect to nail hole locations identified in an intraoperative fluoroscope X-ray image. Audio controller 1914 controls audio played to the surgeon and to other participants via HMD 102 and mobile devices 118. Network message system 1916 passes messages between components of HMD/mobile device client application 806. Collision system 1918 determines when tracked objects and devices collide with each other or with other objects of interest in the environment. Network player controller 1920 controls the playing of surgical video to network connected devices.

### Intramedullary Nail Surgical Procedure

The HMD 102 may register and display a 3D model of the implant (i.e., IM nail) residing in the patient's bone as well as overlay drill trajectory annotations which direct and guide the surgeon to accurately drill a hole the appropriate position and orientation. The fiducial image target marker attached to the insertion handle (see FIG. 11) is used by the surgical navigation system to determine the location of the nail within the medullary canal of the patient. The image target is located on the sensor platform attached to the aiming arm which is, until the procedure is completed, rigidly attached to the IM nail (current standard of care). An inertial measurement unit may also be attached to the insertion handle to obtain additional information about the orientation of the nail within the medullary canal. Similarly, a fiducial image target marker attached to the drill guide (see FIGS. 12A-12C) is used by the surgical navigation system to determine the position and orientation of the drill guide with respect to the holes in the intramedullary nail. The position and orientation of the fiducial image targets on the insertion handle and drill guide are tracked by the system via integrated cameras and other sensors in the HMD AR Viewer. Surgeon voice commands and/or hand gestures also can be accommodated by HMD 102 to enable the surgeon to control the spatial position and/or display of information and graphics appearing around him/her in the 3D physical world.

FIG. 20 is a flow chart illustrating an exemplary process for an IM nail surgical procedure supported by the surgical navigation system described herein. Referring to FIG. 20, the IM nail surgical procedure supported by the surgical navigation system includes the following steps:
2000. insert nail
2001. Take x-ray to determine any nail deflection
2002. Determine incision location, make incision.
2003. Position drill guide at incision location.
2004. Orient drill guide at incision location.
2005. Drill hole.
2006. Insert screw in bone using drill guide.
2007. Take x-ray to confirm screw is in correct location.

Once the position and orientation of the distal screw holes are known, the intersection of the screw hole axes with the bone and skin can be determined. The surgical navigation assists the surgeon in drilling the distal screw holes as follows:

Step 2000: Calibrate Nail Hole Locations. The system can be calibrated before the procedure begins by placing a calibration pin (see FIG. 15) into the nail at the desired hole locations. Once the fiducial image target marker on the calibration pin is recognized, the exact position of the hole location on the nail, as well the orientation of the hole's axis (i.e., its direction in space) can be computed with respect to the image target attached to the insertion handle. As a result, calibration of the system can be completed in a matter of seconds and does not require any prior knowledge of the nail model or its technical specifications.

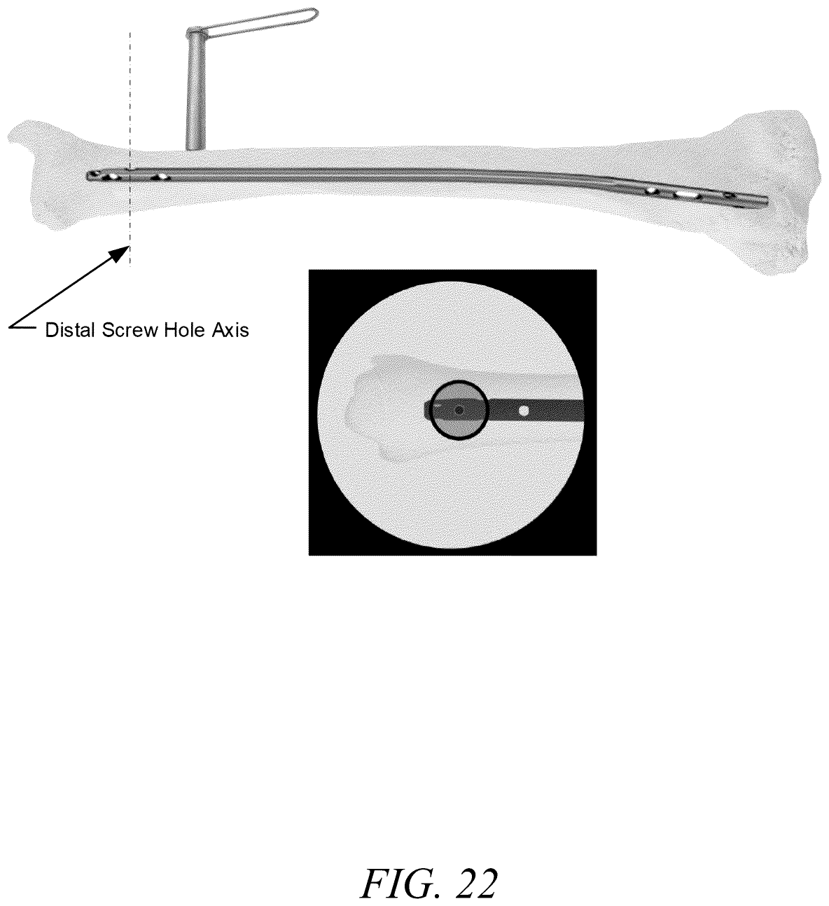

Step 2001: Insert nail. Following appropriate patient preparation, nail selection and identification of anatomic landmarks using fluoroscopy, an incision is made in line with the central axis of the medullary canal. Depending on the anatomy of the patient, this incision can be transpatellar, medial, or lateral parapatellar. The nail entry site is prepared on the ventral edge of the tibial plateau using fluoroscopic guidance. The entry point defines the optimal position of the nail in the intramedullary canal. After inserting the nail in the IM canal, a hologram (i.e., 3D model) of the nail can be displayed in the HMD AR Viewer overlaid on the patient with the distal screw hole locations highlighted and the approximate drill bit entry sites on the patient shown along with the orientation (i.e., axis) of the associated drill holes (see FIG. 22). This virtual “X-ray” image is registered in space (i.e., positioned and aligned) with the “real-world” distal nail residing in the IM canal of the patient.

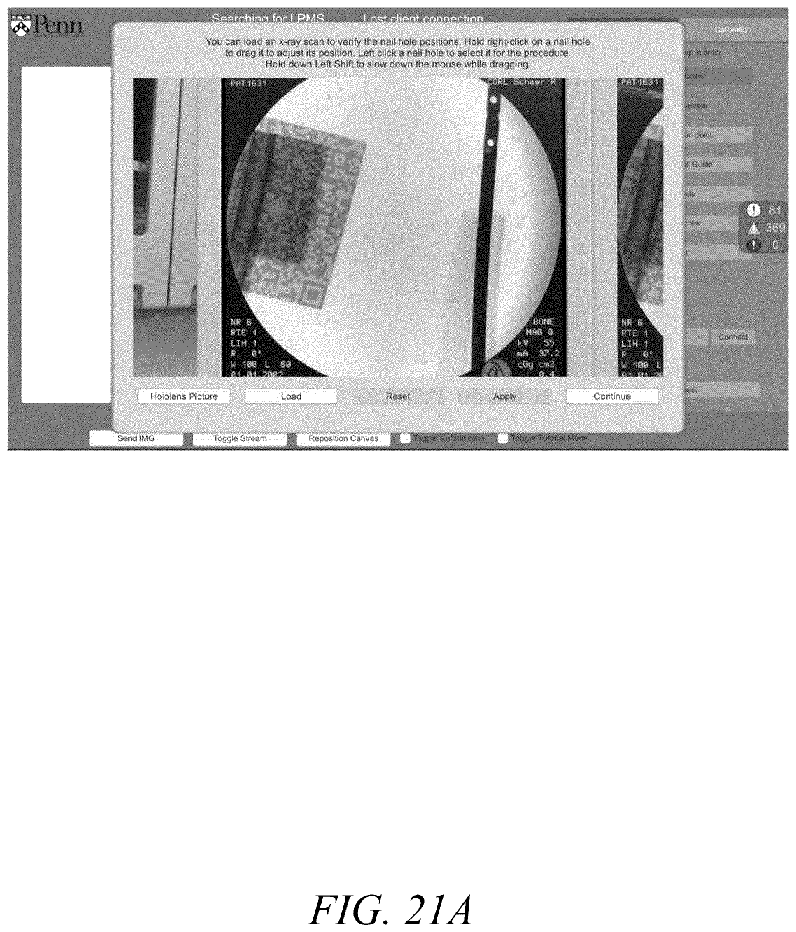

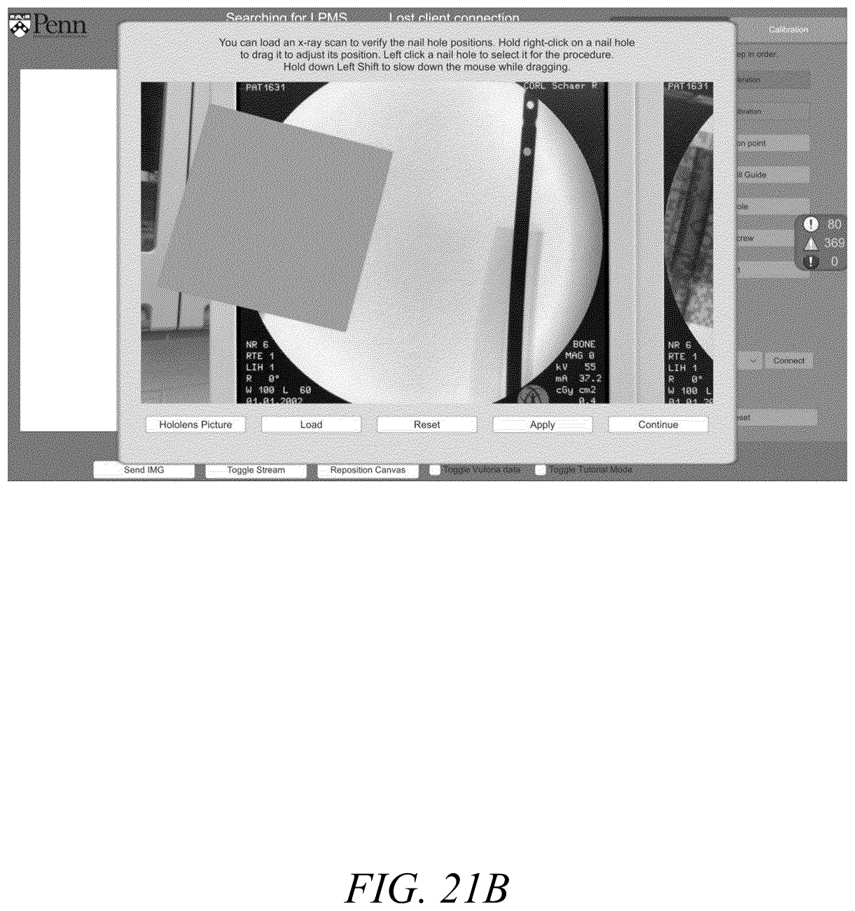

Step 2002: X-Ray to determine nail deflection. Intraoperative images using fluoroscopy, CT or ultrasound are often required during a surgical procedure. This intraoperative data can be imported into the surgical navigation system and used in real-time to update the surgical plan or implant calibration database. For example, the shape of the IM nail may deflect or deform from its original configuration after being inserted into the bone. This problem can be addressed by using an image target on the insertion handle that is also opaque to X-rays so that it appears in the X-ray/fluoroscope image as an image target. This is shown in FIG. 20A. Radiolucent inks or an image target comprised of a printed circuit board (PCB) with copper traces can be used. If such an image target is not available, before the surgical procedures begin, an X-ray of a known X-ray opaque object attached to the operating table can be taken to establish common frame of reference. Using an intraoperative image taken during the surgical procedure that includes the X-ray/fluoroscope opaque image target on the insertion handle (i.e. the image target shown in FIG. 21A), the locations and directions of the distal screw holes axes can be determined using computer vision techniques (i.e., homography). This is shown as the red dot in FIG. 21A. Given these computed locations and directions of the actual locations, offsets can be added to the positions and orientations of the distal screw hole locations and directions obtained from the calibration results of Step 2000 to account for any deflection and/or deformation. This correction is shown in FIG. 18b where the green dot now aligns with the nail hole. The surgical navigation system can import such intraoperative images simply by having the surgeon first look at the intraoperative image displayed on a display screen (for example the fluoroscopic image shown in FIG. 21A). Next, the operator of the desktop application initiates a command in the desktop application that then takes a picture of the display screen (using the built-in Hololens camera) and transfers it over the network to the desktop application for further processing.

FIG. 21A illustrates an intraoperative fluoroscopic display taken by surgeon in operating room showing image target impervious to X-rays, nail hole locations and change in previously calibrated screw hole location due to nail deflection.

FIG. 21B illustrates a computer screen shot of intraoperative procedure showing X-ray hole verification step to address possible nail deformation. The green dot shows changes made to calibrated hole location to account for nail deflection.

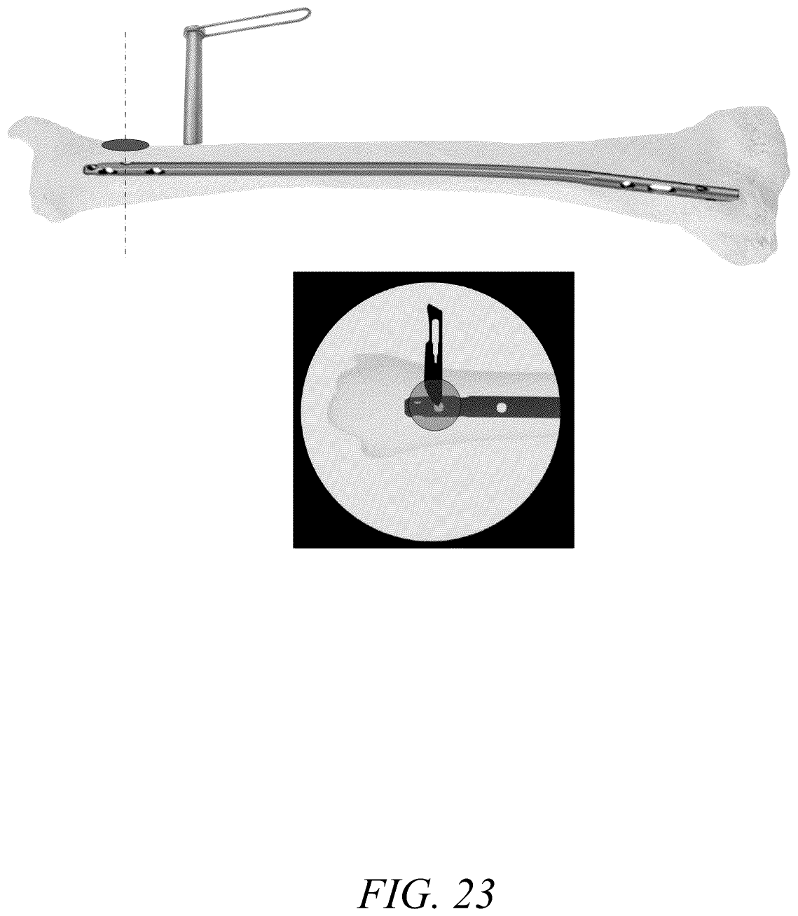

Step 2003: Determine and make incision. In this step the surgeon touches tip of the drill guide to skin in approximate location of distal screw hole. The surgical navigation system computes intersection of screw hole axis with surface of skin and overlays a blue circle at this location. FIG. 22 illustrates the position of the distal screw hole axis. FIG. 23 illustrates the position of the screw hole and the drill guide. The surgeon then makes an incision within area of the blue circle for placement of soft skin protector (i.e., the drill guide).

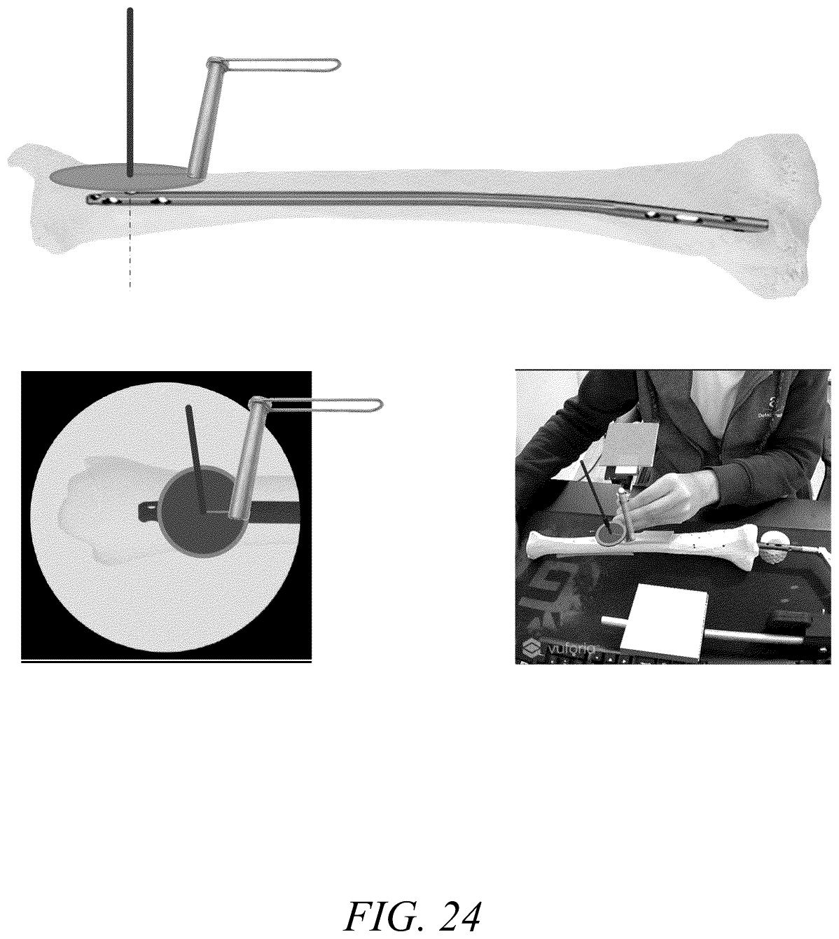

Step 2004: Position drill guide. As shown in FIG. 24, after inserting the bottom of the drill guide in the incision, an animated red circle of decreasing radii appears directing the surgeon to move the bottom of the drill guide towards the distal hole axis (computation details supporting the descriptions in Steps 2001-2006 are provided in the Algorithmic Details section that follows). As the bottom of the drill guide gets closer to the hole axis, a blue collar rises on the hole axis to indicate the current error (i.e., distance to go) between the bottom of the drill guide and the hole axis.

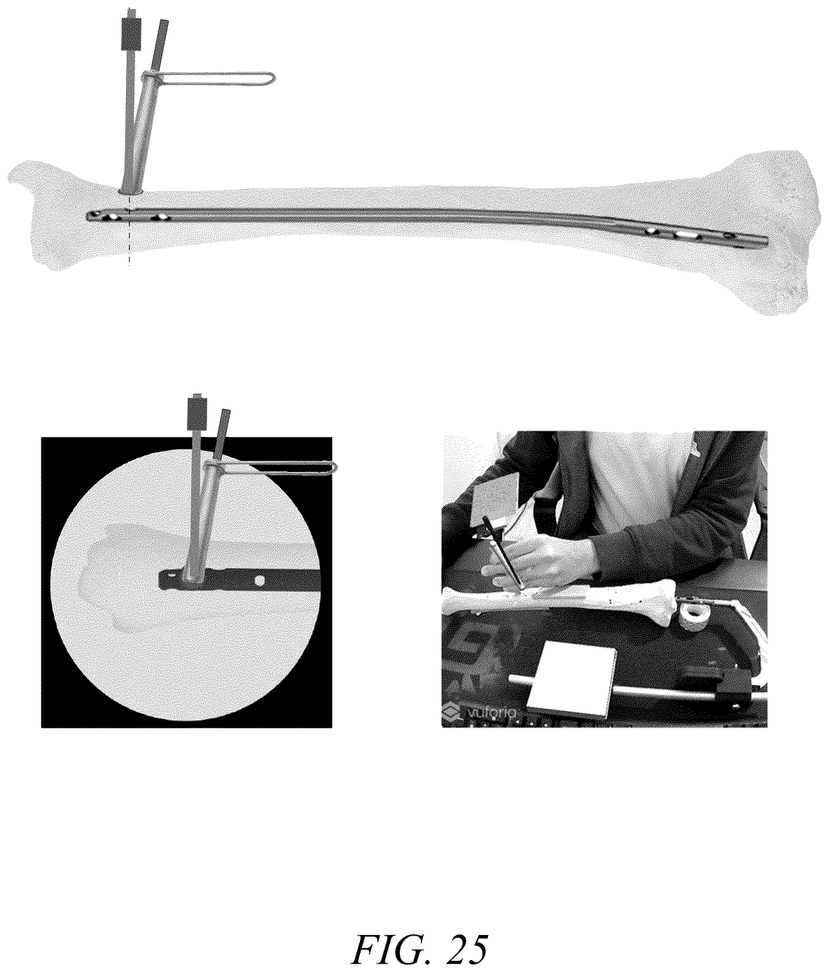

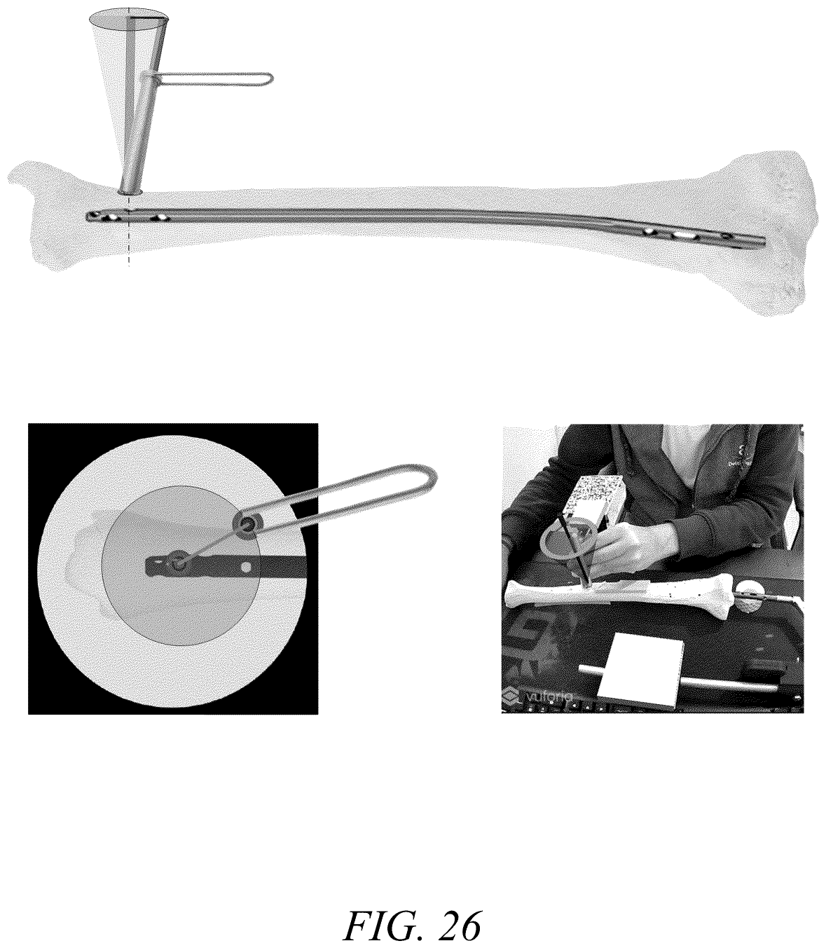

Once the bottom of the drill guide is in the proper location (i.e., at the position of the hole axis), as shown in FIGS. 25 and 26, the animation stops, and the hole axis turns blue. While the user is moving the bottom of the drill guide to the location of the hole axis, the position and orientation of the drill guide is determined using the image target attached to the drill guide. Once the bottom of the drill guide is in the proper position, in order to improve the accuracy of the system as well as eliminate problems with occlusion, only inertial sensor measurements are used to determine the orientation of the drill guide.

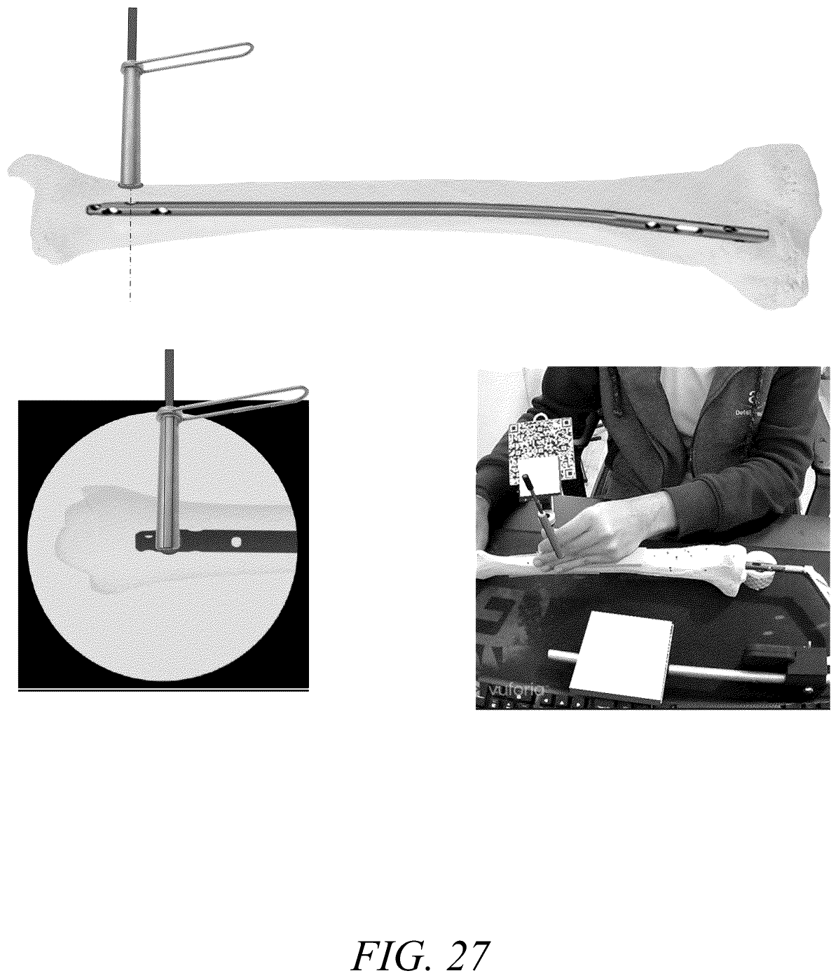

Step 2005: Orient drill guide. As shown in FIG. 27, in this step the surgeon aligns the drill guide axis with the distal screw hole axis by moving the red drill guide axis, so it aligns with the blue hole axis. A red cone associated with the hole axis is used to aid the user in this task. The size of the circle at the top of the red cone indicates the amount of angular error between the two axes. The line connecting the small red and blue circles displayed in the plane of the large circle (shown in the top-down figure below) indicates the most efficient direction to orient the drill guide to reduce the angular error. When the drill guide is properly aligned with the hole axis, the hole axis turns green indicating that the surgeon can now proceed to drill the hole.

The drill trajectories and respective navigation cones are configured and aligned with the distal interlocking screw holes of the IM nail residing in the patient (FIG. 22). The holographic cone annotations shown in FIG. 26 represent the central axis of the drill bit trajectory which aligns with the drill bit entry site and the corresponding screw hole on the other side of the IM nail. The cones and associated graphics are updated in real-time based on the position and orientation data obtained from the sensors attached to the drill sleeve assembly and the position and orientation data from the sensors attached to the insertion handle. Because the insertion handle is rigidly attached to the nail, any movement (i.e., change in position or orientation of the implant system) due to rotation of the nail with respect to the IM canal will be registered by the system. Since the position and orientation of the drill guide is also tracked, the orientation of the drill bit and associated drill trajectory can also be computed in real time.

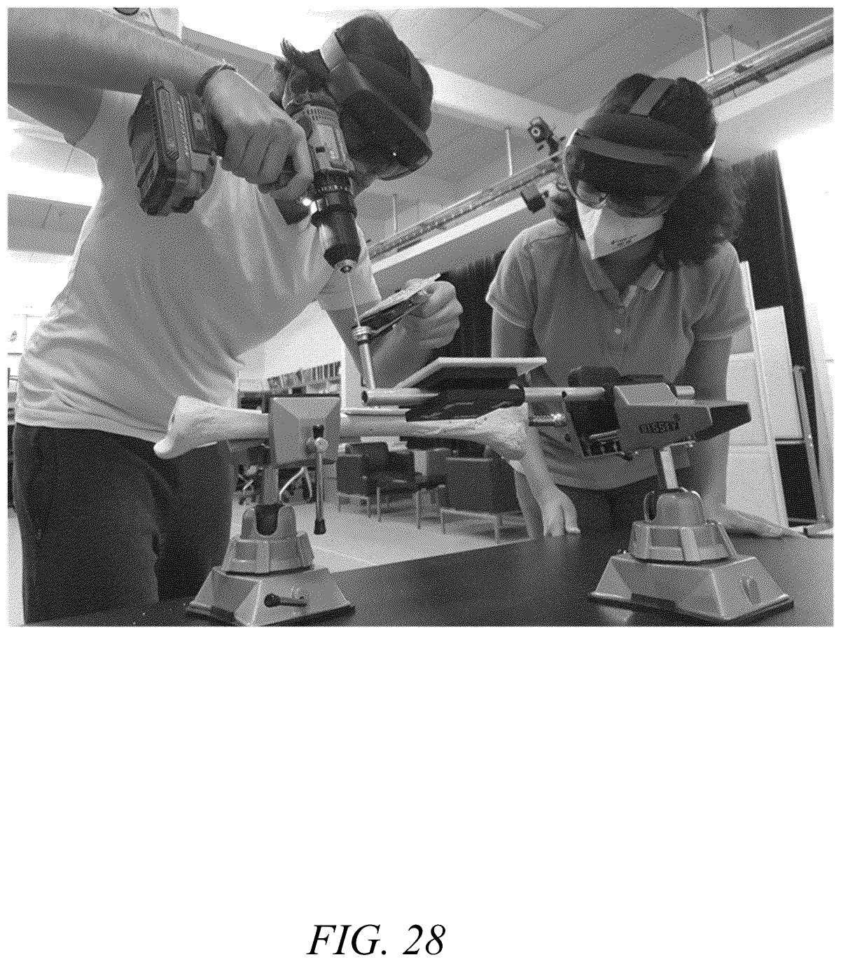

Step 2006: Drill hole. When the blue cylinder and red cylinders are aligned the system displays a green cylinder at the top of the drill. A green circle at the bottom of the drill guide indicates that the drill guide is positioned properly. FIG. 28 illustrates the user drilling a hole when the drill guide is aligned with the virtual drill axis.

The drilling process is then started, and the drill trajectory will be guided by the graphical annotations described above (i.e., cone/drill trajectory line) and the depth of the drill hole can be tracked as described previously using a dedicated sensor or an image target attached to the top of the drill. At the conclusion of the drilling process the drill depth data can be converted to an appropriate screw length and displayed to the surgeon in the HMD AR Viewer.

Step 2007: Insert screw. In this step the surgeon removes the drill bit and inner sleeve (see FIG. 13) from the drill guide assembly and places a screw with the desired length into the outer sleeve of the drill guide. The surgeon then places the drill guide in the proper position (as described in Step 2004) and orients the drill guide (as described in Step 2005) before driving the screw through the near cortex, into the distal screw holes in the nail, then through the far cortex without the need for intraoperative fluoroscopic radiation (i.e., X-rays) which would be required in a “free-hand” approach.

Step 2008: X-ray to confirm screw location (optional). Although the surgeon can complete the procedure for drilling of holes for IM implants without additional intraoperative fluoroscopic guidance (radiation), control fluoroscopic images may be obtained at the end of the procedure to document the final result.

For implant placements, such as external fixator pins or pedicle screws, a marking pin with a fiducial mark (i.e., image target) can be placed at the time of surgery into a pertinent anatomical location. This then allows the position and orientation of the associated anatomy (i.e., bone) to be tracked by the system in real time. As a result, the current system can be extended to provide guidance and support for the surgeon during the drilling process for procedures involving the installation of external fixator pins, pedicle screws and/or other fracture hardware.

### Algorithmic Details

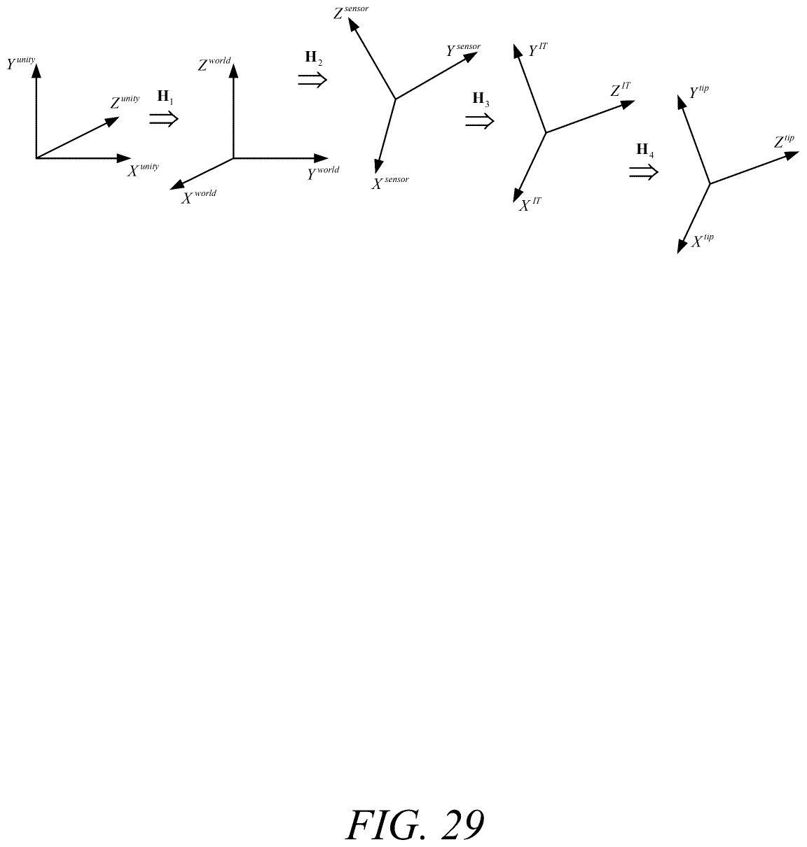

FIG. 29 illustrates coordinate systems and translations of the coordinate systems used by the surgical navigation system described herein. The surgical navigation system requires data representing the position and orientation of various objects and devices (i.e., HoloLens and mobile device viewers, drill guide, drill, insertion handle, etc.) to be transformed between the four coordinate systems shown above where:
{X^{unity} Y^{unity} Z^{unity}} represents the unity coordinate system (left-handed)
{X^{world} Y^{world} Z^{world}} represents the world coordinate system (right-handed)
{X^{sensor} Y^{sensor} Z^{sensor}} represents the LPMS sensor coordinate system (right-handed)
{X^{IT} Y^{IT} Z^{IT}} represents the coordinate system associated with the image target (IT) attached to the drill guide (left-handed)
{X^{tip} Y^{tip} Z^{tip}} represents the coordinate system associated with the tip of the drill guide (left-handed)

In what follows, the general form of the homogenous transformation matrices used takes the form:

$$H = [ \begin{matrix}R & d \\ 0 & 1\end{matrix} ]$$

where R is a 3×3 rotation matrix representing orientation and d is a 3×1 displacement vector representing position (or translation)

and where the inverse of a homogeneous transformation matrix H can be computed as:

$${( H )}^{- 1} = [ \begin{matrix}{R}^{T} & - {R}^{T} d \\ 0 & 1\end{matrix} ]$$

In the surgical navigation system, the position and orientation of the image target attached to the drill guide can be computed in the unity coordinate system using the sequence of transforms shown below:

$${H}_{1 T}^{unity} = {H}_{w o r l d}^{unity} {H}_{s e n s o r}^{w o r l d} {H}_{IT}^{s e n s o r}$$

In the next section it is shown how to compute

$${H}_{w o r l d}^{unity} and {H}_{s e n s o r}^{w o r l d} .$$

Given knowledge of

$${H}_{IT}^{unity} ,$$

which represents the position and orientation of the image target in the Unity coordinate system (obtained from an image target tracking system such as Vuforia), the transform

$${H}_{IT}^{s e n s o r}$$

which represents the orientation of the LMPS sensor with respect to the drill guide can be computed as,

$${H}_{IT}^{s e n s o r} = {( {H}_{w o r l d}^{unity} {H}_{s e n s o r}^{w o r l d} )}^{1} {H}_{IT}^{unity} = {( {H}_{s e n s o r}^{w o r l d} )}^{- 1} {( {H}_{w o r l d}^{unity} )}^{- 1} {H}_{IT}^{unity}$$

where

$${H}_{w o r l d}^{unity}$$

is constant matrix representing how to convert from world to unity coordinates and

$${H}_{s e n s o r}^{w o r l d}$$

is obtained from the LMPS sensor data. The calculation of

$${H}_{IT}^{s e n s o r}$$

above only has to be done once (i.e. as part of the calibration process) since the position and orientation of the LMPS sensor with respect to the drill guide image target does not change during the system operation (since the sensor is physically mounted in drill guide).

Given knowledge of

$${H}_{w o r l d}^{unity} , {H}_{s e n s o r}^{w o r l d} {H}_{IT}^{s e n s o r} and {H}_{t i p}^{I T}$$

(which is determined as part of the drill guide calibration process), the position and orientation of the distal end of the drill guide (i.e. the “tip”) can be computed in the unity coordinate system using the following sequence of transforms,

$${H}_{t i p}^{unity} = {H}_{w o r l d}^{unity} {H}_{s e n s o r}^{w o r l d} {H}_{IT}^{s e n s o r} {H}_{t i p}^{IT}$$

Knowing

$${H}_{tip}^{unity}$$

(i.e., the position and orientation of the tip of the drill guide), the system can now determine errors in position and orientation of the drill guide tip with respect to the nail hole position and axis orientation. These position and alignment errors are what drive the graphical representations shown in FIGS. 23-25.

Given the position of the tip of the drill guide in Unity world coordinates

$$( {p}_{tip}^{Unity} )$$

allows the position of the tip to be computed with respect to the image target attached to the drill guide

$$( {p}_{tip}^{IT} )$$

as follows:

$${p}_{tip}^{IT} = {( {H}_{IT}^{U n i t y} )}^{- 1} {p}_{tip}^{U nity}$$

The orientation of the tip of the drill guide with respect to the image target can be computed during the calibration phase. Given knowledge of the orientation of the calibration pin image target with respect to Unity coordinates and the orientation of the drill guide image target with respect to Unity coordinates, the orientation of the tip of the drill guide with respect to the drill guide image target is:

$${R}_{p i n}^{U nity} = {R}_{I T}^{U nity} {R}_{p i n}^{I T}$$

$${R}_{p i n}^{I T} = {( {R}_{I T}^{Unity} )}^{- 1} {R}_{p i n}^{U nity}$$

Finally, the homogeneous transformation matrix representing the position and orientation of the tip of the drill guide with respect to the image target is:

$${H}_{tip}^{IT} = [ \begin{matrix}{R}_{pin}^{IT} & {p}_{tip}^{IT} \\ 0 & 1\end{matrix} ]$$

Once the system enters LPMS sensor mode the position of the image target is fixed while the orientation of the drill guide is determined by the LPMS sensor. Therefore, the transformation matrix representing a cylindrical graphical oriented along the y-axis of the calibration pin with respect to Unity coordinates would be:

$${cylinder}^{unity} = {H}_{w o r l d}^{unity} {H}_{s e n s o r}^{w o r l d} {H}_{IT}^{s e n s o r} {H}_{tip}^{IT} = {H}_{w o r l d}^{unity} {H}_{s e n s o r}^{w o r l d} {H}_{IT}^{s e n s o r} [ \begin{matrix}{R}_{pin}^{IT} & {p}_{tip}^{IT} \\ 0 & 1\end{matrix} ] {cylinder}^{tip} ,$$

where cylinder^{tip} is a 3D model of a cylinder represented in local coordinates at the tip of the drill guide and cylinder^{unity} representing the position and orientation of the cylinder in unity world coordinates.

### Conversion of LMPS Sensor Orientation to Unity Orientation

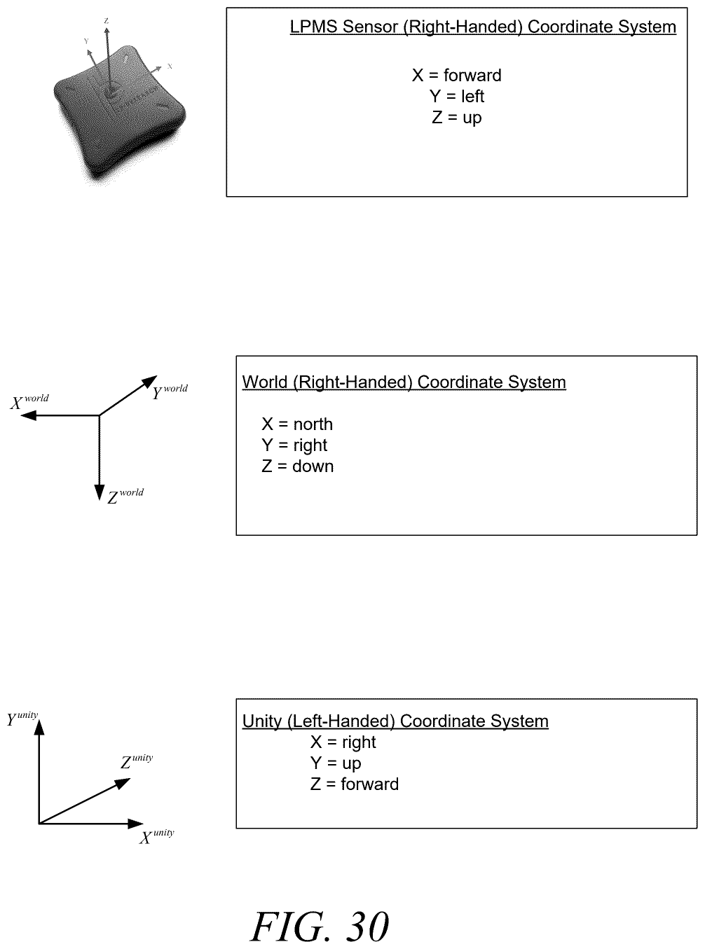

FIG. 30 is a schematic diagram of an LPMS sensor and a translation of the coordinate system used by the LPMS sensor, the real world, and Unity. As can be seen, the LMPS sensor and the world use a right-handed coordinate system while Unity uses a left-handed coordinate system. As a result, vectors produced by the LMPS sensor with respect to the world coordinate system can be converted to Unity coordinates using the following mapping:

$${X}^{unity} = - {X}^{w o r l d}$$

$${Y}^{unity} = - {Z}^{w o r l d}$$

$${Z}^{unity} = - {Y}^{w o r l d}$$

$$In terms of {H}_{w o r l d}^{unity} = [ \begin{matrix}{R}_{world}^{unity} & {d}_{world}^{unity} \\ 0 & 1\end{matrix} ] ,$$

$$where {R}_{w o r l d}^{unity} = [ \begin{matrix}- {X}^{w o r l d} & - {Z}^{w o r l d} & - {Y}^{w o r l d}\end{matrix} ] = [ \begin{matrix}- 1 & 0 & 0 \\ 0 & 0 & - 1 \\ 0 & - 1 & 0\end{matrix} ] and$$

$${d}_{w o r l d}^{unity} = [ \begin{matrix}0 \\ 0 \\ 0\end{matrix} ]$$

A quaternion q can be thought of as an angle-axis representation of orientation where:

$$q = [ \begin{matrix}angle \\ axis\end{matrix} ] = [ \begin{matrix}cos ( \frac{θ}{2} ) \\ sin ( \frac{θ}{2} ) \hat{a}\end{matrix} ] = [ \begin{matrix}w \\ x \\ y \\ z\end{matrix} ] = [ \begin{matrix}cos ( \frac{θ}{2} ) \\ sin ( \frac{θ}{2} ) {a}_{x} \\ sin ( \frac{θ}{2} ) {a}_{y} \\ sin ( \frac{θ}{2} ) {a}_{z}\end{matrix} ]$$

where â is a unit vector representing axis of rotation

To convert quaternion rotations from the LPMS sensor to Unity, two steps are involved:
1. Map the quaternion axis from world coordinates to Unity coordinates.
2. Since a change from right-handed to left-handed coordinates is involved, the direction of positive rotation also goes from counterclockwise to clockwise, so the angle needs to be negated.

Since

$$cos ( \frac{- θ}{2} ) = cos ( \frac{θ}{2} ) and sin ( \frac{- θ}{2} ) = - sin ( \frac{θ}{2} )$$

this is the same as flipping the axis of rotation by negating the x, y, and z components

Given the Unity and world coordinate systems shown above:

| Axis Direction | Unity | World | Alternate Mapping |
|---|---|---|---|
| forward | Z | −Y | Y |
| up | Y | −Z | Z |
| right | X | −X | X |

Combining the two steps (i.e., changes in coordinate mapping and angle negation) yields the ConvertQuatToUnity function:

| Quaternion ConvertQuatToUnity(Quaternion q_input) | |
|---|---|
| { | |
| Quaternion q_output; | |
| q_output.w = q_input.w; | |
| q_output.x = q_input.x; | // −( LHS x = − RHS x ) |
| q_output.y = q_input.z; | // −( LHS y = − RHS z ) |
| q_output.z = q_input.y; | // −( LHS z = − RHS y) |

return output;
}

### Drill Guide Position and Orientation Indicator Calculations

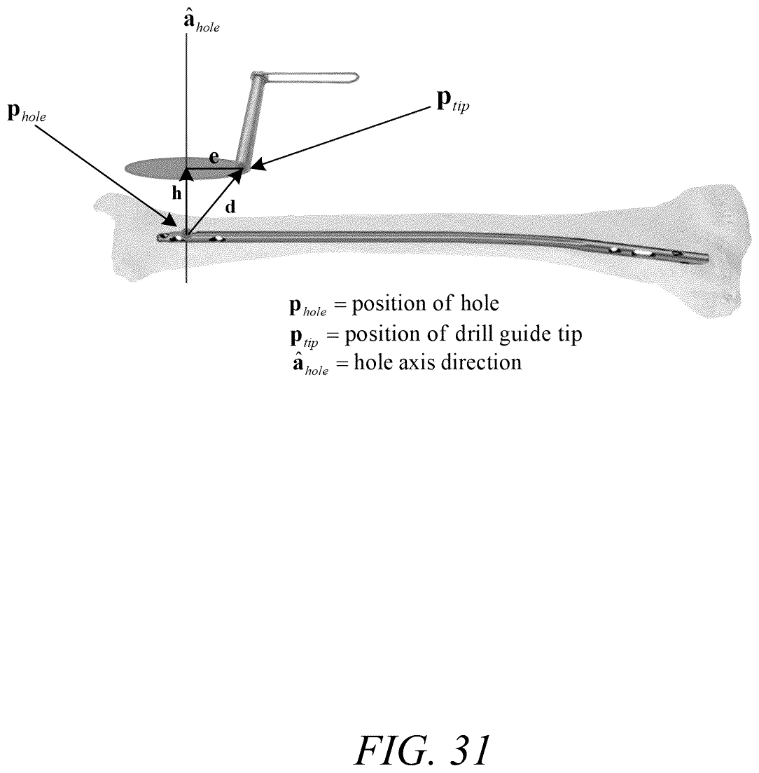

FIG. 31 illustrates distances used in calculating drill guide position and orientation.

Distance of drill guide tip to hole (d)

$$d = ( {p}_{tip} - {p}_{h o l e} )$$

Height of drill guide tip over hole (h).

$$h = ( d \cdot \hat{a} ) \hat{a}$$

h is used to determine the plane of the red error circle that is orthogonal to â

Distance of drill guide tip from hole axis (e).

$$e = ( d - h )$$

∥e∥ defines the radius of the red error circle representing the distance of the drill guide tip from the hole axis.

The extended reality-based surgical navigation system described herein can provide the surgeon with real-time guidance of many common orthopedic surgical procedures from a first-person perspective, without the need for time consuming surgical instrument registration and/or high cost, equipment intensive optical systems or electromagnetic tracking systems.

This is accomplished by:
using commercially available HMD AR viewer devices (such as the Microsoft HoloLens), smart mobile devices (such as Android phones), and sensors attached to existing implant systems (such as insertion handles for intramedullary nails, insertion handles for minimally invasive plate osteosynthesis, pedicle screw placements, etc., that are rigidly attached to the implant system during the surgical procedure);
creating a self-referencing system-No need to set up external cameras or electromagnetic sensor tracking systems. In addition, the Hololens implementation is hands free and consistent with current surgical procedures. Using a self-referencing system allows one hand to hold the drill guide, while the other hand holds the drill;
using computer vision image targets that can be tracked in both optical and fluoroscopic X-ray images, which provides the ability to recalibrate the nail hole locations to address nail and deflection and deformation that takes place during the insertion process using fluoroscopic X-ray images obtained as a normal part of the surgical procedure;
using IMU orientation sensor data in place of orientation estimates obtained from optically-based methods to improve tracking performance and address issues of occlusion; and.
using low cost, off-the-shelf wireless sensors, smart screws (as described above) inserted into the implant system during the surgical procedure and/or optically trackable pins (i.e., smart pins) associated with anatomical landmarks inserted at the beginning of a surgical procedure. Using smart pin markers addresses the shortcomings of current anatomical tracking methodologies which place markers (i.e., image targets) on the surface of the patient which are often subject to movement during manipulation of the soft tissues during a surgical procedure.

In addition:
data associated with smart pin markers can be used by the system to register 3D models of a patient's anatomy (i.e., bones) obtained through preoperative imaging with the actual patient's anatomy at the time of the surgery using intraoperative (fluoroscopy, CT, ultrasound) imaging;
combining position and orientation data from smart pin markers with position and orientation data obtained from the smart drill guide enables the surgical plan, drill trajectories and associated guiding graphical annotations to be updated in real-time to accommodate changes in the patient's anatomy which can often occur during a surgical procedure (for example, during correction of scoliotic spine)
the proposed system can be controlled by an operator using the desktop application or intraoperatively by the surgeon using simple voice commands;
additional users (medical students, nurses, technicians) wearing HMD AR viewer devices or mobile AR devices can also assist and/or participate in the surgical procedure. For example, in a training and education application a senior surgeon might virtually “markup” the surgical area with hand-drawn lines, text and other annotations, using the touch screen or alternatively a pen-based interface of their mobile AR Viewer device as shown in FIG. 7, to provide additional instructions, guidance and direction to a surgeon in training; and.
although CT images are of high quality (i.e., high resolution), CT imaging also exposes patients and physicians to large amounts of ionizing radiation, which causes cancer. On the other hand, ultrasound images can be captured in real time and expose patients and physicians to no radiation. However, for augmented reality applications, the low-quality of ultrasonic imagery (i.e., noisy, and low resolution) limits their use in registering previously computed 3D models of a patient's anatomy with the actual patient anatomy. Using machine learning techniques, it is possible to train a convolutional neural network to produce high resolution, CT quality output images given low resolution, ultrasound images as input. Use of such a trained neural network with the proposed system would allow 3D models of a patient's anatomy to be registered with their actual anatomy in real time without the need to expose the patient to harmful radiation.

As described above, integrating the proposed AR surgical navigation system with a variety of imaging modalities results in a system that produces real-time surgical guidance that significantly decreases:
1. operating time,
2. radiation to both the patient and the operating team, and
3. the need for revision of problematic screw trajectories.

In addition, the use of just in time calibration for determining screw hole locations, the ability to recalibrate to address nail deflection/deformation using intraoperative X-rays taken as a normal part of the procedure, and the ability to easily interface to existing implant systems makes the surgical navigation system agnostic to any commercially available intramedullary nailing solutions, thereby enhancing its utility and commercial viability.

Furthermore, this system opens unprecedented training and teaching opportunities. Providing trainees with HMD or mobile AR devices in or outside the operating theater allows such users to experience the procedure from both their own point of view as well as the point of view of the lead surgeon.

The concepts of the extended reality surgical navigation system developed and disclosed herein can be expanded to other implant systems in the fields of reconstructive spine, pelvic and joint surgery. The ability of software configuration and integration of this system into intraoperative imaging systems (i.e., robotic assisted imaging, robotic assisted ultrasound, or robotic assisted surgery) make it a platform technology for real-time surgical navigation in general.

### Results

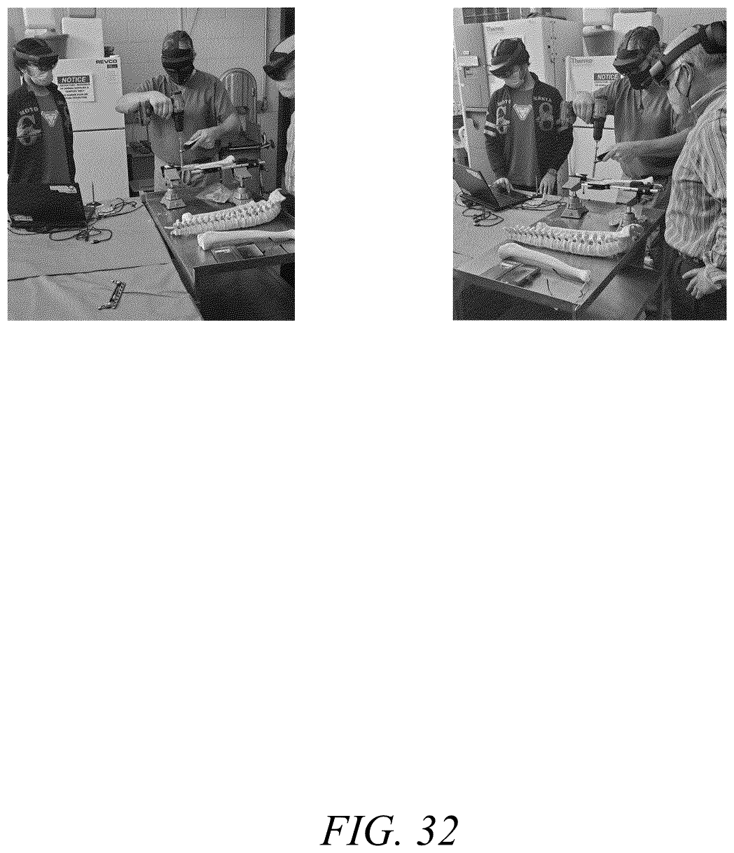

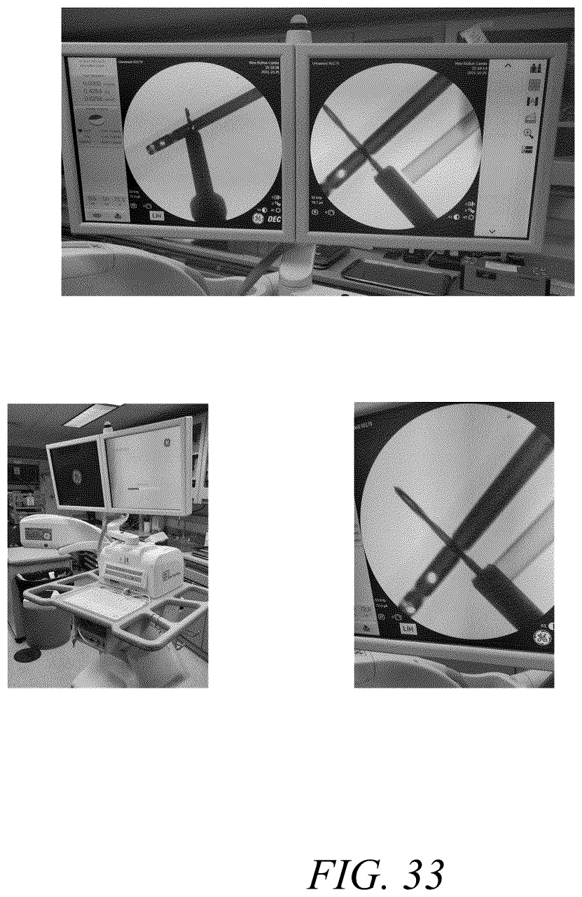

The surgical navigation system described herein was tested and the user was successfully able to drill a hole through a saw bone using the graphical annotations provided by the surgical navigation on the first try. FIG. 32 includes images from the test. FIG. 33 illustrates fluoroscopic control images obtained after the procedure showing the drill bit passing through both sides of the nail screw holes.

### Advancements and Additional Uses

The surgical navigation system described herein includes a commercializable product that has been developed out of the AR enhanced computer assisted surgery system platform developed under this disclosure. The surgical navigation system addresses a clearly defined unmet clinical need for long bone fracture care when using intramedullary nailing systems. The value proposition for the surgical navigation system is that it delivers an improved workflow of distal interlocking screw placement while reducing intraoperative revision rates and exposure to radiation for the surgeon and the patient.

In addition to the uses described above, the surgical navigation system described herein may also be used for percutaneous placement of pedicle screws for the hip. The surgical navigation system may also be used for an interventional radiology system (i.e., transforaminal intrathecal catheter placement in severely deformed scoliosis patients (i.e., EOS-TIS, SMA)).

While the examples described above relate to using the extended reality surgical navigation system to facilitate drilling through a bone, such as a long bone, and through pre-formed screw holes in an intramedullary nail, the subject matter described herein is not limited to these examples. The extended reality surgical navigation system and associated methods described herein can be used to facilitate insertion, placement, fixation, removal, manipulation, or other operation on a surgical object, such as a surgical implant or a surgical tool, including, but not limited to, orthopedic surgical implants or tools.

Although specific examples and features have been described above, these examples and features are not intended to limit the scope of the present disclosure, even where only a single example is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed in this specification (either explicitly or implicitly), or any generalization of features disclosed, whether or not such features or generalizations mitigate any or all of the problems described in this specification. Accordingly, new claims may be formulated during prosecution of this application (or an application claiming priority to this application) to any such combination of features. In particular, with reference to the appended claims, features from dependent claims may be combined with those of the independent claims and features from respective independent claims may be combined in any appropriate manner and not merely in the specific combinations enumerated in the appended claims.

1. A system comprising:
an extended reality head mountable display (HMD); and
a computer system comprising at least one processor, wherein the computer system is configured for performing operations comprising:
receiving camera images while a surgeon is wearing the HMD and performing a surgical procedure on a subject;
identifying at least one image feature depicted in the images;
tracking a location of at least one surgical object using the image feature; and
sending one or more visual indicators to the HMD to display a target location for the surgical object to the surgeon for the surgical procedure, wherein the target location includes a location external to the subject for the surgeon to use the surgical object to perform the surgical procedure on the subject.

2. The system of claim 1, the operations further comprising:
receiving one or more additional images of the subject from a medical imaging system external to the HMD or the HMD; and
recalibrating, using the additional images, the target location to address deflection or deformation or both of a surgically implanted device resulting from an insertion process.

3. The system of claim 2 wherein the one or more additional images comprise fluoroscopy images.

4. The system of claim 1, wherein the surgical object comprises a drill guide and tracking the location of at least one surgical object comprises tracking a location associated with a fixation device located within the subject and a location associated with the drill guide, and wherein the operations comprise determining the target location based on the location associated with the fixation device and the location of the drill guide.

5. The system of claim 4 wherein the fixation device comprises an intramedullary nail, the location associated with the fixation device includes a location of a distal screw hole in the intramedullary nail, and the target location includes a drilling location for the drill guide for a drill bit to pass through the distal screw hole.

6. The system of claim 5 wherein the fixation device comprises an intramedullary nail, the location associated with the fixation device includes a location of a distal screw hole in the intramedullary nail, and the target location includes a drilling location for the drill guide for a drill bit to pass through the distal screw hole

7. The system of claim 1, the operations further comprising:
displaying one or more surgical scene images on one or more mobile devices used by other participants in the surgical procedure;
receiving at least one annotation of the surgical scene images from one of the other participants; and
sending the annotation to the HMD to display to the surgeon for the surgical procedure.

8. The system of claim 1, wherein tracking the location of at least one surgical device comprises receiving and calibrating sensor data from at least one inertial measurement unit (IMU) orientation sensor or other self-referencing sensor.

9. The system of claim 1, wherein the operations comprise executing a pre-operative planning mode and creating a customized surgical plan based on pre-operative subject image data.

10. The system of claim 1, wherein the operations comprise data recording and logging to enable the surgical procedure to be replayed and/or viewed at a later point in time.

11. A method comprising:
receiving images from an extended reality head mountable display (HMD) while a surgeon is wearing the HMD and performing a surgical procedure on a subject;
identifying at least one computer vision image target depicted in the images;
tracking a location of at least one surgical object using the computer vision image target; and
sending one or more visual indicators to the HMD to display a target location for the surgical object to the surgeon for the surgical procedure, wherein the target location includes a location external to the subject for the surgeon to use the surgical object to perform the surgical procedure on the subject.

12. The method of claim 11, comprising:
receiving one or more additional images of the subject from a medical imaging system external to the HMD or the HMD; and
recalibrating, using the additional images, the target location to address deflection or deformation or both of a surgically implanted device resulting from an insertion process.

13. The method of claim 12 wherein the one or more additional images comprise fluoroscopy images.

14. The method of claim 11, wherein the surgical object comprises a drill guide and tracking the location of at least one surgical object comprises tracking a location associated with a fixation device located within the subject and a location associated with the drill guide, and wherein the operations comprise determining the target location based on the location associated with the fixation device and the location of the drill guide.

15. The method of claim 14 wherein the fixation device comprises an intramedullary nail, the location associated with the fixation device includes a location of a distal screw hole in the intramedullary nail, and the target location includes a drilling location for the drill guide for a drill bit to pass through the distal screw hole.

16. The method of claim 15 wherein the fixation device comprises an intramedullary nail, the location associated with the fixation device includes a location of a distal screw hole in the intramedullary nail, and the target location includes a drilling location for the drill guide for a drill bit to pass through the distal screw hole

17. The method of claim 11, comprising:
displaying one or more surgical scene images on one or more mobile devices used by other participants in the surgical procedure;
receiving at least one annotation of the surgical scene images from one of the other participants; and
sending the annotation to the HMD to display to the surgeon for the surgical procedure.

18. (canceled)

19. The method of claim 11, wherein the operations comprise executing a pre-operative planning mode and creating a customized surgical plan based on pre-operative subject image data.

20. The method of claim 11, wherein the operations comprise data recording and logging to enable the surgical procedure to be replayed and/or viewed at a later point in time.

21. A non-transitory computer readable medium storing executable instructions that when executed by at least one processor of a computer control the computer to perform operations comprising:
receiving images from an extended reality head mounted display (HMD) while a surgeon is wearing the HMD and performing a surgical procedure on a subject;
identifying at least one computer vision image target depicted in the images;
tracking a location of at least one surgical object using the computer vision image target, wherein the target location includes a location external to the subject for the surgeon to use the surgical object to perform the surgical procedure on the subject; and
sending one or more visual indicators to the HMD to display a target location for the surgical device to the surgeon for the surgical procedure.
